SEQUENCE LISTING (57 sequences)

SEQ ID NO: 1 - protein, 21 aa, artificial
  CD8 alpha signal peptide
MALPVTALLLPLALLLHAARP

SEQ ID NO: 2 - protein, 20 aa, artificial
  Alternative signal peptide
METDTLLLWVLLLWVPGSTG

SEQ ID NO: 3 - protein, 16 aa, artificial
  Fc gamma RIII alpha hinge
GLAVSTISSFFPPGYQ

SEQ ID NO: 4 - protein, 45 aa, artificial
  CD8 alpha hinge
TTTPAPRPPTPAPTIASQPLSLRPEACRPAAGGAVHTRGLDFACD

SEQ ID NO: 5 - protein, 231 aa, artificial
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, artificial
  CD8 alpha transmembrane domain
IYIWAPLAGTCGVLLLSLVITLYC

SEQ ID NO: 7 - protein, 27 aa, artificial
  4-1BB transmembrane domain
IISFFLALTSTALLFLLFFLTLRFSVV

SEQ ID NO: 8 - protein, 42 aa, artificial
  4-1BB stimulatory domain
KRGRKKLLYIFKQPFMRPVQTTQEEDGCSCRFPEEEEGGCEL

SEQ ID NO: 9 - protein, 112 aa, artificial
  CD3 zeta signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, artificial
  Linker
GGGGSGGGGSGGGGS

SEQ ID NO: 11 - protein, 127 aa, artificial
  CD123 VH
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 12 - protein, 113 aa, artificial
  CD123 VL
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 13 - protein, 499 aa, artificial
  123CAR
MALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGGSGGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 120 aa, artificial
  CS1 VH
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 15 - protein, 107 aa, artificial
  CS1 VL
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 16 - protein, 486 aa, artificial
  CS1CAR
MALPVTALLLPLALLLHAARPQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 120 aa, artificial
  CLL1 VH
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSS

SEQ ID NO: 18 - protein, 107 aa, artificial
  CLL1 VL
DIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIK

SEQ ID NO: 19 - protein, 487 aa, artificial
  CLL1CAR
MALPVTALLLPLALLLHAARPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 124 aa, artificial
  CD22 VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 21 - protein, 107 aa, artificial
  CD22 VL
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 22 - protein, 490 aa, artificial
  CD22CAR
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 378 aa, Homo sapiens
  CD123
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 24 - protein, 287 aa, artificial
  CD123 extracellular domain
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 25 - protein, 536 aa, artificial
  CD123-Fc
MEWSWVFLFFLSVTTGVHSTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 26 - protein, 335 aa, Homo sapiens
  CS1
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 27 - protein, 204 aa, artificial
  CS1 extracellular domain
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 28 - protein, 453 aa, artificial
  CS1-Fc
MEWSWVFLFFLSVTTGVHSSGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 29 - protein, 232 aa, artificial
  CLL1
MSEEVTYADLQFQNSSEMEKIPEIGKFGEKVHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 30 - protein, 201 aa, artificial [sequence omitted]

SEQ ID NO: 31 - protein, 450 aa, artificial
  CLL1-Fc
MEWSWVFLFFLSVTTGVHSHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREAGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 847 aa, Homo sapiens
  CD22
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 33 - protein, 668 aa, artificial
  CD22 extracellular domain
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 34 - protein, 916 aa, artificial
  CD22-Fc
MEWSWVFLFFLSVTTGVHSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMADKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 630 aa, Homo sapiens
  MSLN
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 36 - protein, 292 aa, artificial
  MSLN extracellular domain
EVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQ

SEQ ID NO: 37 - protein, 534 aa, artificial
  MSLN-Fc
MEWSWVFLFFLSVTTGVHSEVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 264 aa, Homo sapiens
  MUC1
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 39 - protein, 135 aa, artificial
  MUC1 extracellular domain
SGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 40 - protein, 385 aa, artificial
  MUC1-Fc
MEWSWVFLFFLSVTTGVHSSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGGGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 41 - protein, 221 aa, artificial
  murine Fc region
CKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 42 - protein, 925 aa, artificial
  TRAC_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 43 - protein, 925 aa, artificial
  TRAC_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 44 - protein, 925 aa, artificial
  CD52_T01-L
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 45 - protein, 925 aa, artificial
  CD52_T01-R
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 46 - protein, 925 aa, artificial
  B2M_T01-L4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 47 - protein, 925 aa, artificial
  B2M_T01-R4
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 48 - protein, 902 aa, artificial
  CS1_T01-L2
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 49 - protein, 902 aa, artificial
  CS1_T01-R2
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 50 - DNA, 49 bp, artificial
  TRAC_T01-target
ttgtcccacagatatccagaaccctgaccctgccgtgtaccagctgaga

SEQ ID NO: 51 - DNA, 49 bp, artificial
  CD52_T01-target
ttcctcctactcaccatcagcctcctggttatggtacaggtaagagcaa

SEQ ID NO: 52 - DNA, 47 bp, artificial
  B2M_T01-target
ttagctgtgctcgcgctactctctctttctggcctggaggctatcca

SEQ ID NO: 53 - DNA, 47 bp, artificial
  CS1_T01-target
tatatcctttggcagctcacaggtgagtccggccggattctcttcca

SEQ ID NO: 54 - protein, 227 aa, artificial
  human Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 225 aa, artificial
  mouse IgG2a Fc
PCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 56 - protein, 760 aa, artificial
  FAP
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 57 - protein, 735 aa, artificial
  FAP extracellular domain
LRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

(19) United States
(12) Patent Application Publication
(10) Pub. No.: US 2023/0228739 A1
(43) Pub. Date: Jul. 20, 2023

(54) Method for determining potency of chimeric antigen receptor expressing immune cells

(71) Applicant: CELLECTIS S.A., Paris (FR)

(72) Inventors: Xenia NAJ, Paris (FR); Anne-Sophie Petit, Paris (FR); Roman Galetto, Paris (FR); Jean-Pierre Cabaniols, Paris (FR)

(21) Appl. No.: 18/002,229

(22) PCT Filed: Jul. 2, 2021
(86) PCT No.: PCT/EP2021/068335

§ 371 Date: Dec. 16, 2022

(30) Foreign Application Priority Data
Jul. 3, 2020 (DK) PA 2020 70460

(51) Int. Cl. G01N 33/50; G01N 33/574; G01N 33/566

(52) U.S. Cl. CPC G01N 33/505; G01N 33/574; G01N 33/566; G01N 2800/7028

(57) ABSTRACT

The invention relates to a new potency assay for characterizing the quality and activity of an immune cell expressing a chimeric antigen receptor, the kit to carry out this assay and uses thereof.

### FIELD OF THE INVENTION

The present invention relates to the field of cell immunotherapy and more particularly to a method of determining the potency of engineered immune cells expressing specific chimeric antigen receptors, themselves useful in the treatment of tumors.

### BACKGROUND

Chimeric antigen receptors (“CAR”) expressing immune cells are cells which have been genetically engineered to express chimeric antigen receptors (CARs) usually designed to recognize specific tumor antigens and kill cancer cells that express the tumor antigen. It is not excluded that the CAR immune cells can activate the immune system to eliminate tumors. These are generally T cells expressing CARs (“CAR-T cells”) or Natural Killer cells expressing CARs (“CAR-NK cells”) or macrophages expressing CARs.

CARs are synthetic receptors consisting of a targeting moiety that is associated with one or more signalling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signalling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010, Blood 116(7):1035-44).

Adoptive immunotherapy, which involves the transfer of autologous or allogeneic antigen-specific T cells generated ex vivo, is a promising strategy to treat viral infections and cancer as confirmed by the increase in the number of CAR-T cells approved by the US Food and Drug Administration (FDA) (e.g. Novartis' anti-CD19 CAR-T tisagenlecleucel (Kymriah™) for the treatment of precursor B-cell acute lymphoblastic leukemia, Kite Pharma's anti-CD19 CAR-T axicabtagene ciloleucel (Yescarta™) for certain types of large B-cell lymphoma in adult patients).

The research and development of CAR-T cell therapy for other malignancies is also in progress and there are currently more than 800 CAR-T clinical trials running.

As more and more investigational new drug applications (INDAs) and new drug applications (NDAs) of immune cells expressing CARs are being submitted, settling the quality control, effectiveness and related regulatory issues of CAR-expressing cells products has become an urgent matter.

For cell-based immunotherapy medicinal products, in particular for the treatment of cancer, guidelines on potency testing exist according to which an appropriately validated potency assay should be based on a defined biological effect as close as possible to the mechanism(s) of action/clinical response. Surrogates for potency may be developed to demonstrate biological activity of the test sample (21 Jul. 2016, EMA/CHMP/BWP/271475/2006 rev.1, Committee for medicinal products for human use (CHMP)).

Current assays for evaluating quality and activity of immune cells expressing CARs make use of a first step of activation of the CAR-T cell by contacting the cell with the antigen for which the CAR is specific, followed by a second step of measurement of cytokine production and/or cell proliferation. In the first step of CAR-T cell activation of current potency assays, the CAR-T cell is placed in contact with the specific antigen either via cells expressing said specific antigen (antigen-expressing cells, tumor cells) or via beads on the surface of which said antigen has been attached (antigen-coated beads), as described, for instance, in WO2018/111340. Xue et al (Journal of Immunotherapy Cancer, 2017, 5:85) disclose a real-time potency assay for CAR-T cells targeting solid and haematological cancer cells comprising a step where the CAR-T cells are activated by contact with target cancer cells.

Basically, existing potency assays are based on antigen presentation (by cell lines or beads) mimicking the antigen presentation occurring at the surface of the tumour cells. This approach is particularly appropriate for screening CARs at the preclinical level, but is not as suitable for developing sensitive standard quality controls as part of GMP manufacturing processes. In particular, for validating CAR-T cells products of pharmaceutical grade, reliable tests (referred to as “potency assays”) are needed to assess immune cells' activity and make sure that clinical cell compositions are physiologically active with comparable levels of CAR activation.

Thus, there still remains a need for improved potency assays, which could, in particular, allow fulfilling the criteria necessary for passing Quality Control and satisfying the Regulatory Agencies' requirements, including high specificity, precision, linearity, robustness, reproducibility.

### SUMMARY

The inventors have developed a new potency assay for characterizing the quality and activity of an immune cell expressing a chimeric antigen receptor, said potency assay is specific, precise, robust, shows a linear response, and constitutes an improvement over the potency assays of the prior art.

One aspect relates to an in vitro method of characterizing the potency of an immune cell expressing a chimeric antigen receptor (CAR), comprising:

(i) Providing an immune cell expressing a chimeric antigen receptor targeting an antigen,

(ii) Stimulating said immune cell by incubating said cell on a ligand-coated support, wherein said support is not a cell and is not a bead,

(iii) Determining the level of activity of the stimulated immune cell,

wherein the ligand coated on the support in step (ii) comprises:
the antigen targeted by said CAR or a fragment of said antigen binding to said CAR; or
an anti-idiotype antibody binding to the antigen-binding domain of said CAR.

Another aspect relates to a kit for determining the potency of an immune cell expressing a chimeric antigen receptor (CAR) targeting an antigen, comprising:

(i) a ligand-coated support, wherein said support is not a cell and is not a bead; and

(ii) one or more reagents for detecting the level of activity of said immune cell;

wherein said CAR binds to said ligand,

wherein said ligand comprises an antigen or fragment thereof comprising at least one epitope(s) targeted by said CAR, or wherein said ligand comprises an anti-idiotype antibody binding to the scFv part of said CAR.

In a further aspect, provided is the use of the method of characterizing the potency of an immune cell expressing a chimeric antigen receptor (CAR) as described herewith for screening for CARs.

A still further aspect relates to an ex vivo method of enrichment of immune cells expressing a chimeric antigen receptor (CAR), comprising:

(i) Providing a population of immune cells comprising immune cells expressing a chimeric antigen receptor targeting an antigen;

(ii) Incubating said population of cells on a ligand-coated support, wherein said support is not a cell and is not a bead and wherein said ligand comprises the antigen targeted by said CAR or a fragment of said antigen binding to said CAR;

whereby the immune cells expressing a CAR targeting said antigen, or a fragment thereof binding to said CAR, have a higher proliferation rate than immune cells that do not express said CAR, resulting in their enrichment.

The present disclosure can be further summarized by the following items:

1. An in vitro method of characterizing the potency of an immune cell expressing a chimeric antigen receptor (CAR), comprising:

(i) Providing an immune cell expressing a chimeric antigen receptor targeting an antigen,

(ii) Stimulating said immune cell by incubating said cell on a ligand-coated support, wherein said support is not a cell and is not a bead,

(iii) Determining the level of activity of the stimulated immune cell,

wherein the ligand coated on the support in step (ii) comprises:
the antigen targeted by said CAR or a fragment of said antigen binding to said CAR; or
an anti-idiotype antibody binding to the antigen-binding domain of said CAR.

2. The method of item 1, wherein said CAR comprises an extracellular antigen-binding domain binding specifically to a target antigen associated with a disease state like a cancer or a viral infection.

3. The method of item 2, wherein said antigen associated with a disease state is a tumor antigen.

4. The method of any one of items 2 to 3, wherein said antigen associated with a disease state is selected from the group consisting of CD123, CD19, CD20, CD22, CD33, 5T4, ROR1, CD38, CS1, BCMA, Flt3, CD70, EGFRvIII, WT1, HSP-70, CLL1, MUC1, ERBB2, MSLN, and FAP.

5. The method of any one of items 1 to 4, wherein said antigen associated with a disease state is CD123.

6. The method of any one of items 1 to 5, wherein said immune cell expresses two or more chimeric antigen receptors binding specifically to different target antigens associated with a cancer.

7. The method of item 6, wherein steps (i) to (iii) are performed with one or more ligand-coated supports comprising (a) a polypeptide comprising any one of the antigens targeted by said CARs, or a fragment thereof binding to said CARs, and/or (b) two or more polypeptides comprising two or more of the antigens targeted by said CARs, or a fragment thereof binding to said CARs.

8. The method of any one of items 1 to 7, wherein said immune cell is selected from the group consisting of a T-cell, a NK-cell, and a macrophage.

9. The method of item 8, wherein said immune cell is a T-cell.

10. The method of any one of items 1 to 9, wherein said immune cell of step (i) is an immune cell obtained from a patient or from a healthy donor, that has been engineered ex vivo to express said CAR.

11. The method of any one of items 1 to 10, wherein the activity which level is determined in step (iii) is cytokine secretion, degranulation, proliferation, or any combination thereof.

12. A kit for determining the potency of an immune cell expressing a chimeric antigen receptor (CAR) targeting an antigen, comprising:

(i) a ligand-coated support, wherein said support is not a cell and is not a bead,

(ii) one or more reagents for detecting the level of activity of said immune cell,

wherein said ligand comprises an antigen targeted by said CAR or a fragment of said antigen binding to said CAR, or

wherein said ligand comprises an anti-idiotype antibody binding to the scFv part of said CAR.

13. The kit according to item 12, wherein said immune cell expresses two or more CARs binding specifically to different target antigens associated with a cancer, wherein said kit comprises two or more ligand-coated supports, and wherein said ligand-coated supports independently comprise (a) a polypeptide comprising any one of the antigens targeted by said CARs, or a fragment thereof binding to said CARs, and/or (b) two or more polypeptides comprising two or more of the antigens targeted by said CARs, or a fragment thereof binding to said CARs

14. The kit according to any one of items 12 to 13, wherein the ligand is attached to the support by passive adsorption through hydrophobic and ionic interactions.

15. The kit according to any one of items 12 to 14, wherein the support is selected from the group consisting of a cell culture plate, a membrane, a matrix, a chip and a glass coverslip.

16. The kit according to any one of items 12 to 15 for determining the potency of an immune cell expressing a CAR targeting CD123, wherein said ligand comprises the extracellular domain of the CD123 antigen of SEQ ID NO. 24, or a fragment thereof comprising at least one epitope binding to said CAR.

17. The kit according to any one of items 12 to 15 for determining the potency of an immune cell expressing a CAR targeting CS1, wherein said ligand comprises the extracellular domain of the CS1 antigen of SEQ ID NO. 27, or a fragment thereof comprising at least one epitope binding to said CAR.

18. The kit according to any one of items 12 to 15 for determining the potency of an immune cell expressing a CAR targeting CLL1, wherein said ligand comprises the extracellular domain of the CLL1 antigen of SEQ ID NO. 30, or a fragment thereof comprising at least one epitope binding to said CAR.

19. The kit according to any one of items 12 to 15 for determining the potency of an immune cell expressing a CAR targeting CD22, wherein said ligand comprises the extracellular domain of the CD22 antigen of SEQ ID NO. 33, or a fragment thereof comprising at least one epitope binding to said CAR.

20. The kit according to any one of items 12 to 15 for determining the potency of an immune cell expressing a CAR targeting FAP, wherein said ligand comprises the extracellular domain of the FAP antigen of SEQ ID NO. 57, or a fragment thereof comprising at least one epitope binding to said CAR.

### BRIEF DESCRIPTION OF THE FIGURES

FIG. 1: Specificity of the potency assay. Measurement of IFNγ secretion by the different CART cells tested: (A) CART123, (B) CARTCS1, (C) CARTCLL1, (D) CART22. CAR-T cells were incubated on a cell culture dish coated with i) their targeted protein ii) no protein or iii) not relevant protein.

FIG. 2: Inter-operator variability of the potency assay. IFNγ secretion measurement by two different operators from the same CAR-T cell batch (either CART123 or CARTCS1) after stimulation on a plate coated with (A) CD123 or (C) CS1, respectively. Coefficient of Variation (CV) percentage between the two operators for CART123 cells (B), or for CARTCS1 cells (D).

FIG. 3: Repeatability of the potency assay. IFNγ secretion measurement after stimulation on an antigen-coated plate in three replicates with (A) CART123 cells, (B) CARTCS1 cells, (C) CARTCLL1 cells, or (D) CART22 cells. The CV is indicated for each triplicate.

FIG. 4: Linearity of the potency assay described herewith using protein-coated wells. Total amount of secreted IFNγ measured for different concentrations of CART cells per ml: (A) CART123 cells, (B) CARTCS1 cells, (C) CARTCLL1 cells, (D) CART22 cells.

FIG. 5. Control potency assay using cells expressing the target protein coated to a plate. IFN-gamma measurement using antigen (CD123) expressing cells incubated with different quantities of two different batches of CART123 cells

FIG. 6: Amount of secreted IFNγ per CAR-T cell for different number of CAR-T cells per ml. (A) CART123 cells, (B) CARTCS1 cells, (C) CARTCLL1 cells, (D) CART22 cells.

FIG. 7: Comparison of different batches of CART123 cells. Measurement of IFNγ per cell obtained with different quantities of different batches of CART123 cells.

FIG. 8: CART123 cell proliferation was measured in presence of IL2 after stimulation with: a CD123-protein coated plate at 4 concentrations of CD123-proteins (W, X, Y, Z), or in control conditions (CART123-cells alone or with an un-relevant protein).

### DETAILED DESCRIPTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Definitions

As used herewith “an antigen associated with a disease state” refers to an antigen present or over-expressed in a given disease. Said disease can be, for instance, a cancer or a viral infection. An antigen associated with a disease state, wherein said disease state is a cancer, i.e. “an antigen associated with a cancer” can be a tumor antigen as defined herewith.

The term “tumor antigen” is meant to cover “tumor-specific antigen” and “tumor associated antigen”. Tumor-Specific Antigens (TSA) are generally present only on tumor cells and not on any other cell, while Tumor-Associated Antigens (TAA) are present on some tumor cells and also present on some normal cells. Tumor antigen, as meant herewith, also refers to mutated forms of a protein, which only appears in that form in tumors, while the non-mutated form is observed in non-tumoral tissues. A “tumor antigen” as defined herewith also includes an antigen associated with the tumor microenvironment and/or the tumor stroma, such as for instance the Fibroblast Activation Protein (FAP) present in tumor stromal fibroblasts.

The term “extracellular antigen-binding domain” as used herein refers to an oligo- or poly-peptide that is capable of binding a specific antigen. Preferably, the domain will be capable of interacting with a cell surface molecule. For example, the extracellular antigen-binding domain may be chosen to recognize an antigen that acts as a cell surface marker on target cells associated with a particular disease state. In a particular instance, said extracellular antigen-binding domain comprises a single chain antibody fragment (scFv) comprising the light (V_{L}) and the heavy (V_{H}) variable fragment of a target-antigen-specific monoclonal antibody joined by a flexible linker. The antigen-binding domain of a CAR expressed on the cell surface of the engineered immune cells described herewith can be any domain that binds to the target antigen and that derives from, for instance, a monoclonal antibody, a recombinant antibody, a human antibody, a humanized antibody, and a functional fragment thereof.

By “chimeric antigen receptor” or “CAR” is generally meant a synthetic receptor comprising a targeting moiety that is associated with one or more signalling domains in a single fusion molecule. As defined herewith, the term “chimeric antigen receptor” covers single chain CARs as well as multi-chain CARs. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T cell cytotoxicity. However, they failed to provide prolonged expansion and anti-tumor activity in vivo. Signalling domains from co-stimulatory molecules including CD28, OX-40 (CD134), and 4-1BB (CD137) have been added alone (second generation) or in combination (third generation) to enhance survival and increase proliferation of CAR modified T cells. CARs are not necessarily only single chain polypeptides, multi-chain CARs are also possible. According to the multi-chain CAR architecture, for instance as described in WO2014039523, the signalling domains and co-stimulatory domains are located on different polypeptide chains. Such multi-chain CARs can be derived from FcεRI, by replacing the high affinity IgE binding domain of FcεRI alpha chain by an extracellular ligand-binding domain such as scFv, whereas the N- and/or C-termini tails of FcεRI beta and/or gamma chains are fused to signal transducing domains and co-stimulatory domains, respectively. The extracellular ligand binding domain has the role of redirecting T-cell specificity towards cell targets, while the signal transducing domains activate the immune cell response.

The term “anti-idiotype antibody” classically describes the antibody that binds to the idiotype of another antibody, usually an antibody drug. In the present application, the term includes an antibody that binds the idiotype of the ScFv part of a CAR. An idiotype can be defined as the specific combination of idiotopes present within an antibody's complement determining regions. A single idiotope is a specific region within an antibody's Fv region which binds to the paratope (antigenic epitope binding site) of a different antibody. Therefore, an idiotope can be considered almost synonymous with an antigenic determinant of an antibody.

By “immune cell” is meant a cell of hematopoietic origin functionally involved in the initiation and/or execution of innate and/or adaptative immune response, such as typically CD45, CD3, CD8 or CD4 positive cells. The immune cell described herewith may be a dendritic cell, killer dendritic cell, a mast cell, macrophage, a natural killer cell (NK-cell), cytokine-induced killer cell (CIK cell), a B-cell or a T-cell selected from the group consisting of inflammatory T-lymphocytes, cytotoxic T-lymphocytes, regulatory T-lymphocytes or helper T-lymphocytes, gamma delta T cells, Natural killer T-cell (“NKT cell).

By “potency” is meant the quantitative measure of the biological activity of cell based immunotherapy products that can be measured using in vivo or in vitro tests. By “potency” of an immune cell is meant a measure of its efficacy or potential efficacy in achieving a desired function or activity. Applied to an immune cell expressing a CAR, the desired function or activity includes targeting or killing another cell, like a tumor cell, expressing on its surface a target antigen specifically recognized by said CAR. Potency can be assessed directly by determination of the effect of the cell on its target (e.g., the effect of a CAR-T cell on a tumor cell in vitro or in vivo). Alternatively, potency can be measured indirectly, for instance, by determining the level of in vitro proliferation of the CAR-expressing immune cell that is specific for the antigen against which the CAR is directed. This measure of potency can then be correlated with, and thus can be considered predictive of, the in vivo properties of the immune cell. As described further in the example section, potency can be expressed in terms of level of cytokine (e.g. IFNγ) secreted per immune cell expressing a relevant CAR and/or in terms of level of proliferation per immune cell expressing a relevant CAR.

The terms “patient” or “subject” and “donor” herein include all members of the animal kingdom including non-human primates and humans.

Antigen-Specific CARs

The immune cells whose potency is determined according to the method and/or using the kits described herewith are endowed with a synthetic chimeric antigen receptor (CAR) targeting an antigen, for instance an antigen associated with a disease state.

In a particular aspect, the immune cells whose potency is determined according to the method and/or using the kits described herewith are endowed with a synthetic CAR which confers them a higher specificity towards a tumor, including specificity towards malignant cells or the tumor microenvironment, or towards infected cells. A recombinant receptor is generally encoded by an exogenous polynucleotide which is introduced into the cell using viral vectors as per one of the transduction steps referred to elsewhere in the current application. A recombinant receptor encoded by an exogenous polynucleotide can also be introduced into the cell in the form of a plasmid or a PCR product.

In one aspect, the CAR expressed by these cells specifically targets an antigen marker at the surface of malignant or infected cells, which further helps said immune cells to destroy these cells in vivo as reviewed by Sadelain M. et al (2013) Cancer Discov. 3(4):388-98.

In another aspect, the CAR expressed by these cells specifically targets an antigen marker at the surface of cells comprised in the tumor stroma, such as the Fibroblast Activation Protein present in tumor stromal fibroblasts.

In general, CAR polypeptides comprise an extracellular antigen-binding domain, a transmembrane domain, and an intracellular domain comprising a costimulatory domain and/or a primary signalling domain, wherein said antigen binding domain binds to the antigen associated with the disease state.

While the method described herewith is not limited to a specific CAR structure, nor on a specific CAR, a nucleic acid that can be used to engineer the immune cells generally encodes a CAR comprising: an extracellular antigen-binding domain that binds to an antigen associated with a disease state, a hinge, a transmembrane domain, and an intracellular domain comprising a stimulatory domain and/or a primary signalling domain. Generally, the extracellular antigen-binding domain is a scFv comprising a Heavy variable chain (VH) and a Light variable chain (VL) of an antibody binding to a specific antigen (e.g., to a tumor antigen) connected via a Linker. The transmembrane domain can be, for example, a CD8α transmembrane domain or a 4-1BB transmembrane domain. The stimulatory domain can be, for example, the 4-1BB stimulatory domain. The primary signalling domain can be, for example, the CD3ζ signalling domain.

TABLE 1
Sequence of different domains typically present in a CAR

| Functional domains | SEQ ID # | amino acid sequence |
|---|---|---|
| CD8α signal | SEQ ID NO.1 | MALPVTALLLPLALLLHAARP |
| peptide (or | | |
| sequence leader) | | |
| Alternative signal | SEQ ID NO.2 | METDTLLLWVLLLWVPGSTG |
| peptide | | |
| FcγRIIIα hinge | SEQ ID NO.3 | GLAVSTISSFFPPGYQ |
| CD8α hinge | SEQ ID NO.4 | TTTPAPRPPTPAPTIASQPLSLRPEACRPAAGGAVH |
| | | TRGLDFACD |
| IgG1 hinge | SEQ ID NO.5 | EPKSPDKTHTCPPCPAPPVAGPSVFLFPPKPKDTL |
| | | MIARTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSRDE |
| | | LTKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGK |
| CD8α | SEQ ID NO. 6 | IYIWAPLAGTCGVLLLSLVITLYC |
| transmembrane | | |
| domain | | |
| 4-1BB | SEQ ID NO. 7 | IISFFLALTSTALLFLLFFLTLRFSVV |
| transmembrane | | |
| domain | | |
| 4-1BB stimulatory | SEQ ID NO. 8 | KRGRKKLLYIFKQPFMRPVQTTQEEDGCSCRFPEE |
| domain | | EEGGCEL |
| CD3ζ signalling | SEQ ID NO. 9 | RVKFSRSADAPAYQQGQNQLYNELNLGRREEYDV |
| domain | | LDKRRGRDPEMGGKPRRKNPQEGLYNELQKDKM |
| | | AEAYSEIGMKGERRRGKGHDGLYQGLSTATKDTY |
| | | DALHMQALPPR |
| Linker | SEQ ID NO. 10 | GGGGSGGGGSGGGGS |

An example of a CAR targeting the CD123 antigen present on tumor cells used to illustrate the present invention is described in Tables 2 and 3 below and in the Example section.

TABLE 2
Sequence of the CD123 VH and VL comprised in the ScFv of the CD123 CAR T
cell illustrated in the Example section

| ScFv | SEQ ID | |
|---|---|---|
| sequences | # | amino acid sequence |
| Heavy variable | SEQ ID | EVKLVESGGGLVQPGGSLSLSCAASGFTFTDYYMSWVRQPPG |
| region | NO. 11 | KALEWLALIRSKADGYTTEYSASVKGRFTLSRDDSQSILYLQMN |
| | | ALRPEDSATYYCARDAAYYSYYSPEGAMDYWGQGTSVTVSS |
| Light variable | SEQ ID | MADYKDIVMTQSHKFMSTSVGDRVNITCKASQNVDSAVAWYQ |
| region | NO. 12 | QKPGQSPKALIYSASYRYSGVPDRFTGRGSGTDFTLTISSVQAE |
| | | DLAVYYCQQYYSTPWTFGGGTKLEIKR |

TABLE 3
Structure of the CD123 CAR-T cell illustrated in the Example section

| CAR | CAR Structure | | | | | | |
|---|---|---|---|---|---|---|---|
| Designation | signal | | | CD8α | CD8α | 4-1BB | CD3ζ |
| 123 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 13) | NO. 1 | NO. 11 | NO. 12 | NO. 4 | NO. 6 | NO. 8 | NO. 9 |

An example of a CAR targeting the CS1 antigen present on tumor cells used to illustrate the present invention is described in Tables 4 and 5 below and in the Example section.

TABLE 4
Sequence of the CS1 VH and VL comprised in the ScFv of the CS1 CAR T cell
illustrated in the Example section

| ScFv | SEQ ID | |
|---|---|---|
| sequences | # | amino acid sequence |
| Heavy variable | SEQ ID | QVQLQQPGAELVRPGASVKLSCKASGYSFTTYWMNWVKQRP |
| region | NO. 14 | GQGLEWIGMIHPSDSETRLNQKFKDKATLTVDKSSSTAYMQLS |
| | | SPTSEDSAVYYCARSTMIATRAMDYWGQGTSVTVSS |
| Light variable | SEQ ID | DIVMTQSQKSMSTSVGDRVSITCKASQDVITGVAWYQQKPGQS |
| region | NO. 15 | PKLLIYSASYRYTGVPDRFTGSGSGTDFTFTISNVQAEDLAVYYC |
| | | QQHYSTPLTFGAGTKLELK |

TABLE 5
Structure of the CS1 CAR-T cell illustrated in the Example section

| CAR | CAR Structure | | | | | | |
|---|---|---|---|---|---|---|---|
| Designation | signal | | | CD8α | CD8α | 4-1BB | CD3ζ |
| CS1 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 16) | NO. 1 | NO. 14 | NO. 15 | NO. 4 | NO. 6 | NO. 8 | NO. 9 |

An example of a CAR targeting the CLL1 antigen present on tumor cells used to illustrate the present invention is described in Tables 6 and 7 below and in the Example section.

TABLE 6
Sequence of the CLL1 VH and VL comprised in the ScFv of the CLL1 CAR T cell
illustrated in the Example section

| ScFv | SEQ ID | |
|---|---|---|
| sequences | # | amino acid sequence |
| Heavy variable | SEQ ID | EVQLQQSGPELVKPGASVKMSCKASGYTFTSYFIHWVKQKPGQ |
| region | NO. 17 | GLEWIGFINPYNDGSKYNEKFKGKATLTSDKSSSTAYMELSSLT |
| | | SEDSAVYYCTRDDGYYGYAMDYWGQGTSVTVSS |
| Light variable | SEQ ID | DIQMTQSPSSLSASLGERVSLTCRATQELSGYLSWLQQKPDGTI |
| region | NO. 18 | KRLIYAASTLDSGVPKRFSGNRSGSDYSLTISSLESEDFADYYCL |
| | | QYAIYPYTFGGGTKLEIK |

TABLE 7
Structure of the CLL1 CAR-T cell illustrated in the Example section

| CAR | CAR Structure | | | | | | |
|---|---|---|---|---|---|---|---|
| Designation | signal | | | CD8α | CD8α | 4-1BB | CD3ζ |
| CLL1 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 19) | NO. 1 | NO. 17 | NO. 18 | NO. 4 | NO. 6 | NO. 8 | NO. 9 |

An example of a CAR targeting the CD22 antigen present on tumor cells used to illustrate the present invention is described in Tables 8 and 9 below and in the Example section.

TABLE 8
Sequence of the CD22 VH and VL comprised in the ScFv of the CD22 CAR T
cell illustrated in the Example section

| ScFv | SEQ ID | |
|---|---|---|
| sequences | # | amino acid sequence |
| Heavy variable | SEQ ID | QVQLQQSGPGLVKPSQTLSLTCAISGDSVSSNSAAWNWIRQSP |
| region | NO. 20 | SRGLEWLGRTYYRSKWYNDYAVSVKSRITINPDTSKNQFSLQL |
| | | NSVTPEDTAVYYCAREVTGDLEDAFDIWGQGTMVTVSS |
| Light variable | SEQ ID | DIQMTQSPSSLSASVGDRVTITCRASQTIWSYLNWYQQRPGKA |
| region | NO. 21 | PNLLIYAASSLQSGVPSRFSGRGSGTDFTLTISSLQAEDFATYYC |
| | | QQSYSIPQTFGQGTKLEIK |

TABLE 9
Structure of the CD22 CAR-T cell illustrated in the Example section

| CAR | CAR Structure | | | | | | |
|---|---|---|---|---|---|---|---|
| Designation | signal | | | CD8α | CD8α | 4-1BB | CD3ζ |
| CD22 CAR | peptide | VH | VL | hinge | TM | IC | CD |
| (SEQ ID | SEQ ID | SEQ ID | SEQ ID | SEQ ID | SEQ ID | SEQ ID | SEQ ID |
| NO. 22) | NO. 1 | NO. 20 | NO. 21 | NO. 4 | NO. 6 | NO. 8 | NO. 9 |

The CAR expressed on the surface of an engineered immune cell described herewith generally binds to specific epitope(s) of an antigen associated to, or mainly expressed in, a pathological cell like a tumor cell, or to an antigen associated with the tumor stroma, or to an antigen associated to a virus. As a result, the CAR-expressing immune cells specifically recognize and bind antigens present on the surface of the target cell and kill the cell. In particular the CAR-expressing immune cells targeting tumor cells can kill the tumor cells.

Many CARs have been described in the art, which can be used to carry out the present method, or to prepare the engineered cells useful in the invention. In particular, such CARs can bind tumor antigens as diverse as one selected from: interleukin 3 receptor subunit alpha (IL3RA also known as CD123); CD19 molecule (CD19); CD1a molecule (CD1a); membrane spanning 4-domains A1 (MS4A1 also known as CD20); CD22 molecule (CD22); CD229 molecule (CD229) CD24 molecule (CD24); CD248 molecule (CD248); CD276 molecule (CD276 or B7H3); CD3 molecule (CD3); CD33 molecule (CD33); CD38 molecule (CD38); CD44v6; CD5 molecule (CD5); CD56 molecule (CD56); CD7 molecule (CD7); CD70 molecule (CD70); CD72; CD79a; CD79b; TNF receptor superfamily member 8 (TNFRSF8 also known as CD30); KIT proto-oncogene receptor tyrosine kinase (CD117); V-set pre-B cell surrogate light chain 1 (VPREB1 or CD179a); adhesion G protein-coupled receptor E5 (ADGRE5 or CD97); TNF receptor superfamily member 17 (TNFRSF17 also known as BCMA); SLAM family member 7 (SLAM F7 also known as CS1); L1 cell adhesion molecule (L1CAM); C-type lectin domain family 12 member A (CLEC12A also known as CLL-1); tumor-specific variant of the epidermal growth factor receptor (EGFRvIII); thyroid stimulating hormone receptor (TSHR); Fms related tyrosine kinase 3 (FLT3); ganglioside GD3 (GD3); Tn antigen (Tn Ag); lymphocyte antigen 6 family member G6D (LY6G6D); Delta like canonical Notch ligand 3 (DLL3); Interleukin-13 receptor subunit alpha-2 (IL-13RA2); Interleukin 11 receptor subunit alpha (IL11RA); mesothelin (MSLN); Receptor tyrosine kinase like orphan receptor 1 (ROR1); Prostate stem cell antigen (PSCA); erb-b2 receptor tyrosine kinase 2 (ERBB2 or Her2/neu); Protease Serine 21 (PRSS21); Kinase insert domain receptor (KDR also known as VEGFR2); Lewis y antigen (LewisY); Solute carrier family 39 member 6 (SLC39A6); Fibroblast activation protein alpha (FAP); Hsp70 family chaperone (HSP70); Platelet-derived growth factor receptor beta (PDGFR-beta); Cholinergic receptor nicotinic alpha 2 subunit (CHRNA2); Stage-Specific Embryonic Antigen-4 (SSEA-4); Mucin 1, cell surface associated (MUC1); mucin 16, cell surface associated (MUC16); claudin 18 (CLDN18); claudin 6 (CLDN6); Epidermal Growth Factor Receptor (EGFR); Preferentially expressed antigen in melanoma (PRAME); Neural Cell Adhesion Molecule (NCAM); ADAM metallopeptidase domain 10 (ADAM10); Folate receptor 1 (FOLR1); Folate receptor beta (FOLR2); Carbonic Anhydrase IX (CA9); Proteasome subunit beta 9 (PSMB9 or LMP2); Ephrin receptor A2 (EphA2); Tetraspanin 10 (TSPAN10); Fucosyl GM1 (Fuc-GM1); sialyl Lewis adhesion molecule (sLe); TGS5; high molecular weight-melanoma-associated antigen (HMWMAA); o-acetyl-GD2 ganglioside (OAcGD2); tumor endothelial marker 7-related (TEM7R); G protein-coupled receptor class C group 5, member D (GPRC5D); chromosome X open reading frame 61 (CXORF61); ALK receptor tyrosine kinase (ALK); Polysialic acid; Placenta-specific 1 (PLAC1); hexasaccharide portion of globoH glycoceramide (GloboH); NY—BR-1 antigen; uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 family member K (LY6K); olfactory receptor family 51 subfamily E member 2 (OR51E2); TCR Gamma Alternate Reading Frame Protein (TARP); Wilms tumor protein (WT1); ETV6-AML1 fusion protein due to 12; 21 chromosomal translocation (ETV6-AML1); sperm autoantigenic protein 17 (SPA17); X Antigen Family, Member 1E (XAGE1E); TEK receptor tyrosine kinase (Tie2); melanoma cancer testis antigen-1 (MAD-CT-1); melanoma cancer testis antigen-2 (MAD-CT-2); Fos-related antigen 1; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Cytochrome P450 1B 1 (CYP1B 1); CCCTC-Binding Factor (Zinc Finger Protein)-Like (BORIS); Squamous Cell Carcinoma Antigen Recognized By T Cells 3 (SART3); Paired box protein Pax-5 (PAXS); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1).

CARs of particular interest in the method described herewith comprise an extracellular binding domain directed against an antigen selected from CD123, CD19, CD20, CD22, CD33, 5T4, ROR1, CD38, CS1, BCMA, Flt3, CD70, EGFRvIII, WT1, HSP-70, CLL1, MUC1, ERBB2, MSLN, and FAP. Such CARs can have the structure described in WO2016120216.

More particularly, CARs expressed by the immune cells on which the methods and kits described herewith can apply comprise an extracellular binding domain directed against an antigen selected from CD123, CD22, CS1, CLL1, MUC1, and MSLN.

Other CARs useful in the method described herewith include CARs comprising an extracellular binding domain directed against an antigen associated with the tumor microenvironment and/or the tumor stroma, such as for instance FAP present in tumor stromal fibroblasts.

Thus, of interest in the methods and kits described herewith are CARs comprising an extracellular binding domain directed against FAP antigen.

Method of Characterizing the Potency of an Immune Cell Expressing a Chimeric Antigen Receptor

In a first aspect, it is described an in vitro method of characterizing the potency of an immune cell expressing a chimeric antigen receptor (CAR), comprising:

(i) Providing an immune cell expressing a chimeric antigen receptor targeting an antigen,

(ii) Stimulating said immune cell by incubating said cell on a ligand-coated support, wherein said support is not a cell and is not a bead,

(iii) Determining the level of activity of the stimulated immune cell,

wherein the ligand coated on the support in step (ii) comprises:
the antigen targeted by said CAR or a fragment of said antigen binding to said CAR; or
an anti-idiotype antibody binding to the antigen-binding domain of said CAR.

Also provided herewith is an in vitro method of characterizing the potency of an immune cell expressing at least one chimeric antigen receptor (CAR), comprising:

(i) Providing an immune cell expressing at least one chimeric antigen receptor targeting an antigen associated with a disease state;

(ii) Stimulating said immune cell by incubating said cell on a ligand-coated support, wherein said support is not a cell and is not a bead;

(iii) Determining the level of activity of the stimulated immune cell;

wherein the ligand coated on the support in step (ii) comprises:
the antigen(s) targeted by said CAR(s) or a fragment of said antigen(s) binding to said CAR(s); or
at least one anti-idiotype antibody binding to the antigen-binding domain of said CAR(s).

Engineered CAR-Expressing Immune Cell

The method and kits described herewith can be applied to any immune cell genetically engineered to express a synthetic chimeric antigen receptor, in particular a chimeric antigen receptor targeting an antigen associated with a disease state such as a tumor antigen or a viral antigen.

In a more particular instance, the genetically engineered immune cell expresses one or more CARs targeting an antigen associated with a cancer such as a tumor-specific antigen, a tumor-associated antigen and/or an antigen associated with the tumor microenvironment and/or the tumor stroma.

In another instance, the genetically engineered immune cell expresses one of more

CARs targeting an antigen selected from the group consisting of CD123, CD19, CD20, CD22, CD33, 5T4, ROR1, CD38, CS1, BCMA, Flt3, CD70, EGFRvIII, WT1, HSP-70, CLL1, MUC1, ERBB2, MSLN, and FAP.

In one instance, said immune cell is an immune cell, such as a T-cell, engineered to express a CD123 CAR.

In another instance, said immune cell is an immune cell, such as a T-cell, engineered to express a CS1 CAR.

In another instance, said immune cell is an immune cell, such as a T-cell, engineered to express a CLL1 CAR.

In another instance, said immune cell is an immune cell, such as a T-cell, engineered to express a CD22 CAR.

In another instance, said immune cell is an immune cell, such as a T-cell, engineered to express a MSLN CAR.

In another instance, said immune cell is an immune cell, such as a T-cell, engineered to express a MUC1 CAR.

In another instance, said immune cell is an immune cell, such as a T-cell, engineered to express a FAP CAR.

In another instance, said immune cell is an immune cell, such as a T-cell, engineered to express more than one CAR.

Especially, said immune cell can express two or more chimeric antigen receptors binding specifically to different target antigens, for instance different target antigens associated with a cancer.

Alternatively, said immune cell can express two or more chimeric antigen receptors binding specifically to different epitopes of the same target antigen, for instance different epitopes of a target antigen associated with a cancer.

Generally, said immune cell expresses 1, 2, 3, 4 or 5 CARs binding specifically to different target antigens or to different epitopes of a target antigen.

Said immune cell can be, for instance, a dendritic cell, killer dendritic cell, a mast cell, a macrophage, a NK-cell, a cytokine-induced Killer (CIK) cell, a B-cell or a T-cell selected from the group consisting of inflammatory T-lymphocytes, cytotoxic T-lymphocytes, regulatory T-lymphocytes or helper T-lymphocytes, gamma delta T cells, NKT cells and tumor infiltrating lymphocytes (TIL).

In a particular instance, the immune cell engineered to express a CAR is selected from the group consisting of a T-cell, a NK-cell, and a macrophage.

In a more particular instance, the immune cell expressing a CAR is a T-cell, for instance a cytotoxic T cell.

In a general instance, said immune cell is comprised in a population of cells, such as a population of immune cells, in particular a population of T-cells, a population of NK-cells, and/or a population of macrophages.

Said genetically engineered immune cells which are assessed using the method described herewith can be generated using any one of the various methods allowing the expression of a CAR on the surface of said immune cell.

Stable expression of CARs in said immune cells can be achieved using, for example, viral vectors (e.g., lentiviral vectors, retroviral vectors, Adeno-Associated Virus (AAV) vectors) or transposon/transposase systems or plasmids or PCR products integration. Other approaches include direct mRNA electroporation.

A population of immune cells to be engineered is generally extracted from a patient's or healthy donor's blood by apheresis and further engineered to express a chimeric antigen receptor at their surface. Alternatively, a population of immune cells to be engineered could derive from cord blood cells or from stem cells, which are further engineered to express a chimeric antigen receptor at their surface.

Said CAR-expressing immune cells may derive from a patient's or a compatible donor's immune cells which have been engineered to express a specific CAR at their surface.

Said CAR-expressing immune cells can also derive from stem cells, such as iPS cells, originating from such patient or compatible donor or from tumor infiltrating lymphocytes (TIL).

In other aspects, said CAR-expressing immune cells are so-called “off the shelf” immune cells compositions, whereby immune cells not specially belonging to the patient to be treated have been engineered to express a CAR and to become suitable for use in an allogeneic therapeutic treatment.

By “allogeneic” is meant that the cells originate from a donor, or are produced and/or differentiated from stem cells in view of being infused into patients having a different haplotype.

Such immune cells are generally engineered to be less alloreactive and/or become more persistent with respect to their patient host. More specifically, the method of engineering allogeneic immune cells can comprise the step of reducing or inactivating TCR expression into T-cells, or into the stem cells to be derived into T-cells. This can be obtained by different sequence specific-reagents, such as by gene silencing or gene editing techniques (nuclease, base editing, RNAi . . . ).

The applicant has formerly made available robust protocols and gene editing strategies to produce allogeneic therapeutic grade T-cells from PBMCs, especially by providing very safe and specific endonuclease reagents under the form of TALE-nucleases (TALEN®). The production of so-called “universal T-cells”, which are [TCR]^{neg} T-cells from donors was achieved and successfully injected to patients with reduced Graft versus Host Disease (GVhD) (Poirot et al. 2015, Cancer. Res. 75 (18): 3853-3864; Qasim et al., 2017, Science Translational 9(374)). Meanwhile, inactivation of TCR or β2m components in primary T-cells can be combined with the inactivation of further genes encoding checkpoint inhibitor proteins, such as described for instance in WO2014184744.

In further instances, the engineered immune cell can be further modified to confer resistance to at least one immune suppressive drug, such as by inactivating CD52 that is the target of anti-CD52 antibody (e.g.: alemtuzumab), as described for instance in WO2013176915.

In further instances, the engineered immune cell can be further modified to confer resistance to and/or a chemotherapy drug, in particular a purine analogue drug, for example by inactivating DCK as described in WO201575195.

In further instances, the engineered immune cell can be further modified to improve its persistence or its lifespan into the patient, in particular inactivating a gene encoding MHC-I component(s) such as HLA or β2m, such as described in WO2015136001 or by Liu et al. (2017, Cell Res 27:154-157).

In still further instances, the engineered immune cell is mutated to improve its CAR-dependent immune activation, in particular to reduce or suppress the expression of immune checkpoint proteins and/or their receptors thereof, such as PD1 or CTLA4 as described in WO2014184744.

Ligand-Coated Support

In one aspect, the ligand attached to the support as described in the method described herewith comprises an antigen targeted by said CAR or a fragment of said antigen binding to said CAR.

In one instance, the ligand attached to the support as described herewith comprises a fragment of the antigen targeted by said CAR, said fragment comprising at least one epitope binding to said CAR.

In another instance, the ligand attached to the support as described herewith comprises the extracellular domain of the antigen targeted by said CAR, or a fragment thereof comprising at least one epitope binding to said CAR.

An epitope as described above generally comprises about 5 to 10 amino acids from the targeted antigen, which form a domain recognized by, and binding to, the CAR.

In another instance, when the immune cell to be assessed by the method and/or kit described herewith expresses two or more CARs, the method may have to be run independently with different ligands and/or the kit may comprise different ligands. In this instance, a ligand may comprise any one of the antigens or fragments thereof targeted by said CARs as one polypeptide. In this instance, several ligands as defined herewith may be attached on the same support or on separate supports.

In an alternative instance of when the immune cell to be assessed by the method and kit described herewith expresses two or more CARs, one ligand may comprise two or more of the antigens or fragments thereof targeted by said CARs as a fusion protein.

In a particular instance, in particular when the immune cell to be assessed by the method and kit described herewith expresses two or more CARs, the method may have to be run independently for one, two or more ligands as defined above attached to separate supports. For instance, said two or more antigens can be comprised in independent polypeptides attached either one by one or as a group of several ones on separate supports. For instance, one particular antigen, or fragment thereof, can be attached to one support and another antigen, or fragment thereof, can be attached to another support. In that case, the method described herewith may be run independently for each couple of particular CAR and corresponding antigen-coated support. In another instance, one particular antigen, or fragment thereof, can be attached to the same support as another antigen, or fragment thereof.

As used herewith, “separate support” includes using independent supports as well as using different positions of a given support (e.g. different wells of a given cell culture plate).

Thus, in a particular instance of the method described herewith, steps (i) to (iii) are performed with one or more ligand-coated supports, wherein said ligand comprises:
a polypeptide comprising any one of the antigens targeted by said CARs, or a fragment thereof binding to said CARs, and/or
two or more polypeptides comprising two or more of the antigens targeted by said CARs, or a fragment thereof binding to said CARs.

In another particular instance, said two or more antigens are attached to the support as a fusion protein.

In a further instance, the immune cell to be assessed expresses two CARs targeting an antigen associated with a disease state, and the ligand described herewith comprises a fusion polypeptide comprising the antigens targeted by said two CARs, or a fragment thereof binding to said CAR(s).

In a still further instance, the ligand described herewith comprises the CD123 antigen or the extracellular domain of the CD123 antigen, or a fragment thereof comprising at least one epitope binding to the CD123 CAR expressed by the immune cell.

The CD123 antigen can comprise SEQ ID NO. 23, or any variant thereof with at least 95%, at least 98%, at least 99% sequence identity therewith.

In a still further instance, the ligand described herewith comprises the extracellular domain of the CD123 antigen of SEQ ID NO. 24, or any variant thereof with at least 95%, at least 98%, at least 99% sequence identity therewith.

In a still further instance, the ligand described herewith comprises the CS1 antigen, or the extracellular domain of the CS1 antigen, or a fragment thereof comprising at least one epitope binding to the CS1 CAR expressed by the immune cell.

The CS1 antigen can comprise SEQ ID NO. 26 or any variant thereof with at least 95%, at least 98%, at least 99% sequence identity therewith. In a still further instance, the ligand described herewith comprises the extracellular domain of the CS1 antigen of SEQ ID NO. 27, or any variant thereof with at least 95%, at least 98%, at least 99% sequence identity therewith.

In a still further instance, the ligand described herewith comprises the CLL1 antigen, or the extracellular domain of the CLL1 antigen, or a fragment thereof comprising at least one epitope binding to the CLL1 CAR expressed by the immune cell.

The CLL1 antigen can comprise SEQ ID NO. 29, or any variant thereof with at least 95%, at least 98%, at least 99% sequence identity therewith.

In a still further instance, the ligand described herewith comprises the extracellular domain of the CLL1 antigen of SEQ ID NO. 30, or any variant thereof with at least 95%, at least 98%, at least 99% sequence identity therewith.

In a still further instance, the ligand described herewith comprises the CD22 antigen or the extracellular domain of the CD22 antigen, or a fragment thereof comprising at least one epitope binding to the CD22 CAR expressed by the immune cell.

The CD22 antigen can comprise SEQ ID NO. 32 or any variant thereof with at least 95%, at least 98%, at least 99% sequence identity therewith.

In a still further instance, the ligand described herewith comprises the extracellular domain of the CD22 antigen of SEQ ID NO. 33, or any variant thereof with at least 95%, at least 98%, at least 99% sequence identity therewith.

In a still further instance, the ligand described herewith comprises the MSLN antigen or the extracellular domain of the MSLN antigen, or a fragment thereof comprising at least one epitope binding to the MSLN CAR expressed by the immune cell.

The MSLN antigen can comprise SEQ ID NO. 35, or any variant thereof with at least 95%, at least 98%, at least 99% sequence identity therewith.

In a still further instance, the ligand described herewith comprises the extracellular domain of the MSLN antigen of SEQ ID NO. 36, or any variant thereof with at least 95%, at least 98%, at least 99% sequence identity therewith.

In a still further instance, the ligand described herewith comprises the MUC1 antigen or the extracellular domain of the MUC1 antigen, or a fragment thereof comprising at least one epitope binding to the MUC1 CAR expressed by the immune cell.

The MUC1 antigen can comprise SEQ ID NO. 38, or any variant thereof with at least 95%, at least 98%, at least 99% sequence identity therewith.

In a still further instance, the ligand described herewith comprises the extracellular domain of the MUC1 antigen of SEQ ID NO. 39, or any variant thereof with at least 95%, at least 98%, at least 99% sequence identity therewith.

In a still further instance, the ligand described herewith comprises the FAP antigen or the extracellular domain of the FAP antigen, or a fragment thereof comprising at least one epitope binding to the FAP CAR expressed by the immune cell.

The FAP antigen can comprise SEQ ID NO. 56 or the FAP extracellular domain of SEQ ID NO. 57, or any variant thereof with at least 95%, at least 98%, at least 99% sequence identity therewith.

In a still further instance, the ligand described herewith further comprises additional parts having additional utilities, such as amino acid sequences useful to improve the process of production of the ligand, such as amino acid sequences useful to stabilize the ligand, and/or such as amino acid sequences useful for coupling the ligand to the support. In a particular instance, the ligand described herewith further comprises a signal sequence such as the signal sequence comprised between positions 1 and 19 of SEQ ID NO. 25.

In another particular instance, the ligand described herewith further comprises the sequence of a fragment crystallizable region (Fc domain) of an antibody, such as a murine Fc domain of an IgG, such as the Fc sequence of SEQ ID NO. 41 or the Fc sequence of SEQ ID NO. 55, or such as a human Fc domain such as the Fc sequence of SEQ ID NO. 54.

In a still further particular instance that is useful for carrying out the potency assay of a CD123 CAR T cell, the ligand described herewith comprises the amino acid sequence of SEQ ID NO. 25 or amino acids 20 to 536 of SEQ ID NO. 25.

In a still further particular instance that is useful for carrying out the potency assay of a CS1 CAR T cell, the ligand described herewith comprises the amino acid sequence of SEQ ID NO. 28 or amino acids 20 to 453 of SEQ ID NO. 28.

In a still further particular instance that is useful for carrying out the potency assay of a CLL1 CAR T cell, the ligand described herewith comprises the amino acid sequence of SEQ ID NO. 31 or amino acids 20 to 450 of SEQ ID NO. 31.

In a still further particular instance that is useful for carrying out the potency assay of a CD22 CAR T cell, the ligand described herewith comprises the amino acid sequence of SEQ ID NO. 34 or amino acids 20 to 916 of SEQ ID NO. 34.

In a still further particular instance that is useful for carrying out the potency assay of a MSLN CAR T cell, the ligand described herewith comprises the amino acid sequence of SEQ ID NO. 37 or amino acids 20 to 534 of SEQ ID NO. 37.

In a still further particular instance that is useful for carrying out the potency assay of a MUC1 CAR T cell, the ligand described herewith comprises the amino acid sequence of SEQ ID NO. 40 or amino acids 20 to 385 of SEQ ID NO. 40.

In a still further particular instance that is useful for carrying out the potency assay of a FAP CAR T cell, the ligand described herewith comprises the amino acid sequence of SEQ ID NO. 56 or SEQ ID NO. 57.

In a still further particular instance that is useful for carrying out the potency assay of a FAP CAR T cell, the ligand described herewith comprises the amino acid sequence of SEQ ID NO. 57 and the amino acid sequence of a Fc sequence of SEQ ID NO. 41.

In another aspect, the ligand attached to the support as described herewith is an anti-idiotype antibody binding to the antigen-binding domain of said CAR.

An anti-idiotype antibody binds to the idiotype of another antibody or to the idiotype of a CAR, within the ScFv part of said antibody or CAR. An anti-idiotype antibody binds to at least one paratope in the scFv part of the CAR expressed by the immune cell.

An anti-idiotype antibody may thus mimic the antigen targeted by the original antibody or CAR against which said anti-idiotype is directed. An anti-idiotype antibody is different from the antigen. As a consequence, an antibody or CAR which specifically binds a particular antigen will also specifically bind its complementary anti-idiotype antibody.

Methods to prepare antibodies, and in particular anti-idiotype antibodies, are well known to the skilled person.

Typically, antibodies (including anti-idiotypes antibodies) are produced by challenging an animal with the target antigen, in an antigenically active form, and recovering antiserum, or recovering lymphocytes and fusing them with myeloma cells to form hybridomas. Specific antibodies (including anti-idiotype antibodies) can also be selected from a library of antibodies using the widely used phage display technology as described, for instance, in Kretzschmar et al. (Current Opinion in Biotechnology, 2002, 13(6): 598-602), Zhao et al. (PLos One, 2014, 9:9(5)), Tornetta et al. (J. Immunol. Methods, 2007, 328: 34-44), Coelho et al. (Br J Cancer, 2004, 90: 2032-2041), Krishnaswamy et al. (J. Immunol. Methods, 2011, 366: 60-68), Rahman et al. (J. Mol. Recognit, 2011, 24: 631-641).

In one instance, the ligand (such as the antigen targeted by the CAR expressed by the immune cell or a fragment thereof comprising at least one epitope binding to said CAR) is attached to a support, wherein said support is not a cell and is not a bead.

The support can be a solid support.

In alternative aspects, the support can be a gel such as Collagen, Poly-L-Lysine, Poly-D-Lysine, Gelatin, Fibronectin, Matrigel, Laminin.

In particular, said ligand (such as the antigen or fragment thereof as described herewith) is attached to a solid planar support like a cell culture plate (e.g., a multi-well culture plate, a microtiter plate, a microtiter plate comprising 24, 48 or 96 microtiter plate wells), a membrane such as a synthetic membrane (e.g. a nitrocellulose membrane, PVDF membrane, nylon membrane), a matrix (fiber matrix, Sepharose matrix, sugar matrix), a chip (plastic chip, glass chip), a glass coverslip.

There are no restrictions regarding the manner of attaching the ligand to the support.

The ligand can be attached to, or immobilized on, the support by covalent bound(s) or non-covalent bound(s) (e.g., cross linked, via ionic forces, via passive adsorption through hydrophobic and ionic interactions), either directly or indirectly via a molecule itself attached to the plate or via a molecule attached to the polypeptide comprising the antigen.

In a particular instance, the antigen is coupled to a crystallizable fragment (Fc) domain of an antibody, such as an IgG. For instance, the Fc domain can be a murine Fc domain, a rat Fc domain, or a human Fc domain of an IgG. More particularly, the Fc domain is the murine Fc domain of an IgG of SEQ ID NO. 41 or SEQ ID NO. 55, or a human Fc domain of SEQ ID NO. 54. The resulting ligand can be immobilized on a support coated with Protein A or Protein G.

In a further instance, the antigen is coupled to a crystallizable fragment (Fc) domain of an antibody, and further coupled to a biotin label. The resulting ligand can be immobilized on a streptavidin-, avidin- or neutravidin-coated support.

In another instance, the antigen is coupled to a biotin label. The resulting ligand can be immobilized on a streptavidin-, avidin- or neutravidin-coated support.

In another instance, the antigen is coupled to a poly-Histidine label. The resulting ligand can be immobilized on a support coated with anti-His antibody.

Biological Activity of the Stimulated Cell

Any biological activity exhibited by the engineered immune cell expressing a CAR can be determined, including, for instance, cytokine production and secretion, degranulation, proliferation, or any combination thereof.

In a particular instance, the biological activity determined in step (iii) is cytokine secretion, cell proliferation, or both.

Said biological activities can be measured by standard methods well known by the skilled person, in particular by in vitro and/or ex vivo methods.

Secretion of any cytokine can be measured, in particular secretion of IFNγ, TNFα, can be determined. Standard methods to determine cytokine secretion includes ELISA, flow cytometry. These methods are described for instance in Sachdeva et al. (Front Biosci, 2007, 12:4682-95) and Pike et al (2016) (Methods in Molecular Biology, vol 1458. Humana Press, New York, N.Y.).

The level of cytokine secretion measured in the potency assay described herewith can be expressed, for instance, as the maximum level of cytokine (e.g., IFNγ) secreted per CAR-expressing immune cell (e.g., CAR-T cell), e.g. maximum amount of IFNγ secreted per CAR-T cell.

Alternatively, the level of cytokine secretion measured in the potency assay described herewith can be expressed as the total level of cytokine secreted.

For instance, assays to evaluate cytolytic activity include the chromium-51 release assay (CRA) (Holden et al. (1977) Journal of National Cancer Institute 58(3):611-622), alternative non-radioactive assays using bis(acetoxymethyl)-2,2′:6′,2″-terpyridine-6,6″″-dicar-boxylate (BATDA) or carboxyfluorescein diacetate succinimidyl ester (CFSE) fluorescent dye labelling, imaging-based assays measuring apoptosis of target cells using fluorescent probes (Somanchi et al. (2015) PLOS ONE 10(10), e0141074; Mukherjee et al. (2017) Journal of the American Society of Gene Therapy 25(8): 1757-1768), and flow cytometry assays that detect apoptotic target cells (Zaritskaya (2010) Expert Review of Vaccines 9(6):601-616).

To evaluate “degranulation”, standard methods can be used, including for instance CD107a degranulation assay or measurement of secreted Granzyme B or Perforin (such as described in Lorenzo-Herrero et al, Methods Mol Biol, 2019, 1884:119-130; Betts et al. Methods in Cell Biology, 2004, 75:497-512).

To evaluate “proliferation” activity, standard methods can be carried out, which are mainly based on methods involving measurement of DNA synthesis, detection of proliferation-specific markers, measurement of successive cell divisions by the use of cell membrane binding dyes, measurement of cellular DNA content and measurement of cellular metabolism.

A particular instance relates to an in vitro method of characterizing the potency of an immune cell expressing a CAR targeting the CD123 antigen, comprising:

(i) Providing an immune cell expressing a CAR targeting the CD123 antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 24, or the amino acid sequence of SEQ ID NO. 25 or amino acids 20 to 536 of SEQ ID NO. 25, and

wherein said ligand is attached to the cell culture plate via passive adsorption through hydrophobic and ionic interactions.

In another instance is provided an in vitro method of characterizing the potency of an immune cell expressing a CAR targeting the CD22 antigen, comprising:

(i) Providing an immune cell expressing a CAR targeting the CD22 antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 33, or the amino acid sequence of SEQ ID NO. 34 or amino acids 20 to 916 of SEQ ID NO. 34, and

wherein said ligand is attached to the cell culture plate via passive adsorption through hydrophobic and ionic interactions.

In another instance is provided an in vitro method of characterizing the potency of an immune cell expressing a CAR targeting the CS1 antigen, comprising:

(i) Providing an immune cell expressing a CAR targeting the CS1 antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 27, the amino acid sequence of SEQ ID NO. 28 or amino acids 20 to 453 of SEQ ID NO. 28, and

wherein said ligand is attached to the cell culture plate via passive adsorption through hydrophobic and ionic interactions.

In another instance is provided an in vitro method of characterizing the potency of an immune cell expressing a CAR targeting the CLL1 antigen, comprising:

(i) Providing an immune cell expressing a CAR targeting the CLL1 antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 30, or the amino acid sequence of SEQ ID NO. 31 or amino acids 20 to 450 of SEQ ID NO. 31, and

wherein said ligand is attached to the cell culture plate via passive adsorption through hydrophobic and ionic interactions.

In another instance is provided an in vitro method of characterizing the potency of an immune cell expressing a CAR targeting the MSLN antigen, comprising:

(i) Providing an immune cell expressing a CAR targeting the MSLN antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 36, or the amino acid sequence of SEQ ID NO. 37 or amino acids 20 to 534 of SEQ ID NO. 37, and

wherein said ligand is attached to the cell culture plate via passive adsorption through hydrophobic and ionic interactions.

In another instance is provided an in vitro method of characterizing the potency of an immune cell expressing a CAR targeting the MUC1 antigen, comprising:

(i) Providing an immune cell expressing a CAR targeting the MUC1 antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 39, or the amino acid sequence of SEQ ID NO. 40 or amino acids 20 to 385 of SEQ ID NO. 40, and

wherein said ligand is attached to the cell culture plate via passive adsorption through hydrophobic and ionic interactions.

In another instance is provided an in vitro method of characterizing the potency of an immune cell expressing a CAR targeting the FAP antigen, comprising:

(i) Providing an immune cell expressing a CAR targeting the FAP antigen;
(ii) Stimulating said immune cell by incubating said cell on a ligand-coated cell culture plate;
(iii) Determining the level of activity of the stimulated immune cell,

wherein said CAR binds to said ligand,

wherein said ligand comprises the amino acid sequence of SEQ ID NO. 56, or the amino acid sequence of SEQ ID NO. 57, and optionally the Fc amino acid sequence of SEQ ID NO. 41, and

wherein said ligand is attached to the cell culture plate via passive adsorption through hydrophobic and ionic interactions.

In further particular instances, said method applies to immune cells which are T cells.

In one instance of said method, the level of cytokine production, in particular IFNγ production is determined in step (iii) of said method.

In another instance of said method, the level of proliferation is determined in step (iii) of said method.

In a still other instance of said method, the level of both cytokine production and proliferation are determined in one or several step(s) (iii).

In a particular instance, the stimulation of the immune cell in step (ii) of the method described herewith is carried out at about 37° C. for a period of time comprised between 1 and 5 days, for instance about 24 hours or 4 days.

Kit

Another aspect concerns a kit for determining the potency of an immune cell expressing a chimeric antigen receptor (CAR) targeting an antigen, comprising:

(i) a ligand-coated support, wherein said support is not a cell and is not a bead; and
(ii) one or more reagents for detecting the level of activity of said immune cell;

wherein said CAR binds to said ligand,

wherein said ligand comprises an antigen or fragment thereof comprising at least one epitope(s) targeted by said CAR, or wherein said ligand comprises an anti-idiotype antibody binding to the scFv part of said CAR.

In one instance, said kit is for determining the potency of an immune cell expressing a CAR targeting an antigen associated with a disease state like a cancer or a viral infection.

In one instance of the kit described herewith, said antigen is a tumor antigen or a viral antigen.

In a particular instance, said kit is for determining the potency of an immune cell expressing more than one CAR targeting a tumor-specific antigen, a tumor-associated antigen and/or an antigen associated with the tumor microenvironment and/or the tumor stroma.

In another instance, said kit is for determining the potency of an immune cell expressing at least two CARs targeting two different antigens associated with a cancer.

In an alternative instance, said kit is for determining the potency of an immune cell expressing at least two CARs targeting two different epitopes of an antigen associated with a cancer.

Another aspect relates to a kit for determining the potency of an immune cell expressing at least one CAR targeting an antigen associated with a cancer, comprising:

(i) a ligand-coated support, wherein said support is not a cell and is not a bead,
(ii) one or more reagents for detecting the level of activity of said immune cell,

wherein said ligand comprises at least one antigen targeted by said CAR or a fragment of said antigen binding to said CAR.

A still other aspect relates to a kit for determining the potency of an immune cell expressing at least one CAR targeting an antigen associated with a cancer, comprising:

(i) a ligand-coated support, wherein said support is not a cell and is not a bead,
(ii) one or more reagents for detecting the level of activity of said immune cell,

wherein said ligand comprises at least one anti-idiotype antibody binding to the scFv part of said CAR.

Another aspect relates to a kit for determining the potency of an immune cell expressing at least one CAR targeting an antigen associated with the tumor microenvironment and/or the tumor stroma, comprising:

(i) a ligand-coated support, wherein said support is not a cell and is not a bead,
(ii) one or more reagents for detecting the level of activity of said immune cell,

wherein said ligand comprises at least one antigen targeted by said CAR or a fragment of said antigen binding to said CAR.

In one instance, when said kit is for determining the potency of an immune cell expressing two or more CARs binding specifically to different target antigens associated with a cancer, or to different epitopes of an antigen associated with a cancer, said kit may comprise two or more ligand-coated supports, wherein said ligand-coated supports independently comprise (a) a polypeptide comprising any one of the antigens (or epitopes) targeted by said CARs, or a fragment thereof binding to said CARs, and/or (b) two or more polypeptides comprising two or more of the antigens (or epitopes) targeted by said CARS, or a fragment thereof binding to said CARs. Thus, said kit may comprise two or more ligand-coated supports, wherein each ligand comprises a polypeptide comprising an antigen (or epitope) targeted by one of said CARs. In another instance, said kit may comprise two or more ligands comprising a polypeptide comprising an antigen (or epitope) targeted by one of said CARs, wherein said ligands are coated on one or more supports. Said ligands may be attached to the same support or to different supports.

In one alternative instance, when said kit is for use with an immune cell expressing two or more chimeric antigen receptors (CARs) binding different antigens associated with a cancer, or different epitopes of an antigen associated with a cancer, said ligand may comprise a fusion polypeptide comprising the antigens (or epitopes) targeted by said CARs, or a fragment thereof binding to said CARs.

In one instance, the kit described herewith comprises a ligand attached to, or immobilized on, the support by covalent bound(s) or non-covalent bound(s) (e.g., cross linked, via ionic forces, via passive adsorption through hydrophobic and ionic interactions), either directly or indirectly via a molecule itself attached to the plate or via a molecule attached to the polypeptide comprising the antigen.

In a particular instance, the kit described herewith comprises a ligand comprising the antigen coupled to a crystallizable fragment (Fc) domain of an antibody (such as an IgG) and is immobilized on a support coated with Protein A or Protein G.

In a further instance, the kit described herewith comprises a ligand comprising the antigen coupled to a crystallizable fragment (Fc) domain of an antibody, and further coupled to a biotin label. The resulting ligand comprised in the kit can be immobilized on a streptavidin-, avidin- or neutravidin-coated support.

In another instance, the kit described herewith comprises a ligand comprising said antigen coupled to a biotin label, resulting in said ligand being immobilized on a streptavidin-, avidin- or neutravidin-coated support.

According to one aspect of the kit described herewith, the ligand is attached to the support by passive adsorption through hydrophobic and ionic interactions.

According to a further aspect of the kit described herewith, the support is selected among a cell culture plate (e.g., a multi-well culture plate, a microtiter plate, a microtiter plate comprising 24, 48 or 96 microtiter plate wells), a membrane such as a synthetic membrane (e.g. a nitrocellulose membrane, PVDF membrane, nylon membrane), a matrix (fiber matrix, Sepharose matrix, sugar matrix), a chip (plastic chip, glass chip), a glass coverslip.

In another instance is provided a kit for determining the potency of an immune cell expressing a chimeric antigen receptor (CAR) as described herewith, comprising:

(i) an antigen-specific coated cell culture plate,

(ii) one or more reagents for detecting the level of activity of said immune cell,

wherein said CAR is specific for said antigen.

In a further instance, is provided a kit for determining the potency of a CD123 CAR-T cell, the kit comprising:

(i) a CD123 antigen-coated cell culture plate,

(ii) one or more reagents for detecting the level of activity of said CD123 CAR-T cell.

In a further instance, the CD123 antigen comprises the extracellular domain of CD123 of SEQ ID NO. 24, or a fragment thereof comprising at least one epitope binding to said CAR.

In a still further instance, the CD123 antigen is attached to the coated cell culture plate by passive adsorption through hydrophobic and ionic interactions.

In a still further instance, the CD123 antigen is coupled to a murine Fc region and is attached to a Protein A-coated cell culture plate.

In a still further instance, the CD123 antigen is labelled with biotin and is attached to a streptavidin-coated cell culture plate.

In a particular instance, is provided a kit for determining the potency of an immune cell, such as a T cell, expressing a CAR targeting the CD123 antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 24, or the amino acid sequence of SEQ ID NO. 25 or amino acids 20 to 536 of SEQ ID NO. 25, and wherein said ligand is attached to the cell culture plate via passive adsorption through hydrophobic and ionic interactions.

In a still further instance, the level of activity of IFNγ secreted by the CD123-CAR T-cell is determined by ELISA.

In a still further instance, the level of proliferation of the CD123 CAR-T cell is determined by counting the number of viable cells by standard methods in the field.

In another instance, is provided a kit for determining the potency of a CS1 CAR-T cell, the kit comprising:

(i) a CS1 antigen-coated cell culture plate,

(ii) one or more reagents for detecting the level of activity of said CS1 CAR-T cell.

In a further instance, the CS1 antigen comprises the extracellular domain of CS1 of SEQ ID NO. 27, or a fragment thereof comprising at least one epitope binding to said CAR.

In a still further instance, the CS1 antigen is attached to the coated cell culture plate by passive adsorption through hydrophobic and ionic interactions.

In a still further instance, the CS1 antigen is coupled to a murine Fc region and is attached to a Protein A-coated cell culture plate.

In a still further instance, the CS1 antigen is labelled with biotin and is attached to a streptavidin-coated cell culture plate.

In a particular instance, is provided a kit for determining the potency of an immune cell, such as a T cell, expressing a CAR targeting the CS1 antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 27, the amino acid sequence of SEQ ID NO. 28 or amino acids 20 to 453 of SEQ ID NO. 28, and

wherein said ligand is attached to the cell culture plate via passive adsorption through hydrophobic and ionic interactions.

In a still further instance, the level of activity of IFNγ secreted by the CS1-CAR T-cell is determined by ELISA.

In a still further instance, the level of proliferation of the CS1 CAR-T cell is determined by counting the number of viable cells by standard methods in the field.

In a further instance, is provided a kit for determining the potency of a CLL1 CAR-T cell, the kit comprising:

(i) a CLL1 antigen-coated cell culture plate,

(ii) one or more reagents for detecting the level of activity of said CLL1 CAR-T cell.

In a further instance, the CLL1 antigen comprises the extracellular domain of CLL1 of SEQ ID NO. 30, or a fragment thereof comprising at least one epitope binding to said CAR.

In a still further instance, the CLL1 antigen is attached to the coated cell culture plate by passive adsorption through hydrophobic and ionic interactions.

In a still further instance, the CLL1 antigen is coupled to a murine Fc region and is attached to a Protein A-coated cell culture plate.

In a still further instance, the CLL1 antigen is labelled with biotin and is attached to a streptavidin-coated cell culture plate.

In a particular instance, is provided a kit for determining the potency of an immune cell, such as a T cell, expressing a CAR targeting the CLL1 antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 30, or the amino acid sequence of SEQ ID NO. 31 or amino acids 20 to 450 of SEQ ID NO. 31, and

wherein said ligand is attached to the cell culture plate via passive adsorption through hydrophobic and ionic interactions.

In a still further instance, the level of activity of IFNγ secreted by the CLL1 CAR T-cell is determined by ELISA.

In a still further instance, the level of proliferation of the CLL1 CAR-T cell is determined by counting the number of viable cells by standard methods in the field.

In a further instance, is provided a kit for determining the potency of a CD22 CAR-T cell, the kit comprising:

(i) a CD22 antigen-coated cell culture plate,

(ii) one or more reagents for detecting the level of activity of said CD22 CAR-T cell.

In a further instance, the CD22 antigen comprises the extracellular domain of CD22 of SEQ ID NO. 33, or a fragment thereof comprising at least one epitope binding to said CAR.

In a still further instance, the CD22 antigen is attached to the coated cell culture plate by passive adsorption through hydrophobic and ionic interactions.

In a still further instance, the CD22 antigen is coupled to a murine Fc region and is attached to a Protein A-coated cell culture plate.

In a still further instance, the CD22 antigen is labelled with biotin and is attached to a streptavidin-coated cell culture plate.

In a particular instance, is provided a kit for determining the potency of an immune cell, such as a T cell, expressing a CAR targeting the CD22 antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 33, or the amino acid sequence of SEQ ID NO. 34 or amino acids 20 to 916 of SEQ ID NO. 34, and

wherein said ligand is attached to the cell culture plate via passive adsorption through hydrophobic and ionic interactions.

In a still further instance, the level of activity of IFNγ secreted by the CD22 CAR T-cell is determined by ELISA.

In a still further instance, the level of proliferation of the CD22 CAR-T cell is determined by counting the number of viable cells by standard methods in the field.

In a further instance, is provided a kit for determining the potency of a MSLN CAR-T cell, the kit comprising:
(i) a MSLN antigen-coated cell culture plate,
(ii) one or more reagents for detecting the level of activity of said MSLN CAR-T cell.

In a further instance, the MSLN antigen comprises the extracellular domain of MSLN of SEQ ID NO. 36, or a fragment thereof comprising at least one epitope binding to said CAR.

In a still further instance, the MSLN antigen is attached to the coated cell culture plate by passive adsorption through hydrophobic and ionic interactions.

In a still further instance, the MSLN antigen is coupled to a murine Fc region and is attached to a Protein A-coated cell culture plate.

In a still further instance, the MSLN antigen is labelled with biotin and is attached to a streptavidin-coated cell culture plate.

In another particular instance, is provided a kit for determining the potency of an immune cell, such as a T cell, expressing a CAR targeting the MSLN antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 36, or the amino acid sequence of SEQ ID NO. 37 or amino acids 20 to 534 of SEQ ID NO. 37, and

wherein said ligand is attached to the cell culture plate via passive adsorption through hydrophobic and ionic interactions.

In a still further instance, the level of activity of IFNγ secreted by the MSLN-CAR T-cell is determined by ELISA.

In a still further instance, the level of proliferation of the MSLN CAR-T cell is determined by counting the number of viable cells by standard methods in the field.

In a further instance, is provided a kit for determining the potency of a MUC1 CAR-T cell, the kit comprising:
(i) a MUC1 antigen-coated cell culture plate,
(ii) one or more reagents for detecting the level of activity of said MUC1 CAR-T cell.

In a further instance, the MUC1 antigen comprises the extracellular domain of MUC1 of SEQ ID NO. 39, or a fragment thereof comprising at least one epitope binding to said CAR.

In a still further instance, the MUC1 antigen is attached to the coated cell culture plate by passive adsorption through hydrophobic and ionic interactions.

In a still further instance, the MUC1 antigen is coupled to a murine Fc region and is attached to a Protein A-coated cell culture plate.

In a still further instance, the MUC1 antigen is labelled with biotin and is attached to a streptavidin-coated cell culture plate.

In a particular instance, is provided a kit for determining the potency of an immune cell, such as a T cell, expressing a CAR targeting the MUC1 antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 39, or the amino acid sequence of SEQ ID NO. 40 or amino acids 20 to 385 of SEQ ID NO. 40,

wherein said ligand is attached to the cell culture plate via passive adsorption through hydrophobic and ionic interactions.

In a still further instance, the level of activity of IFNγ secreted by the MUC1 CAR T-cell is determined by ELISA.

In a still further instance, the level of proliferation of the MUC1 CAR-T cell is determined by counting the number of viable cells by standard methods in the field.

In a further instance, is provided a kit for determining the potency of a FAP CAR-T cell, the kit comprising:
(i) a FAP antigen-coated cell culture plate,
(ii) one or more reagents for detecting the level of activity of said FAP CAR-T cell.

In a further instance, the FAP antigen comprises the extracellular domain of FAP of SEQ ID NO. 57, or a fragment thereof comprising at least one epitope binding to said CAR.

In a still further instance, the FAP antigen is attached to the coated cell culture plate by passive adsorption through hydrophobic and ionic interactions.

In a still further instance, the FAP antigen is coupled to a murine Fc region and is attached to a Protein A-coated cell culture plate.

In a still further instance, the FAP antigen is labelled with biotin and is attached to a streptavidin-coated cell culture plate.

In a particular instance is provided a kit for determining the potency of an immune cell, such as a T cell, expressing a CAR targeting the FAP antigen, comprising:

(i) a ligand-coated cell culture plate; and
(ii) one or more reagents for detecting the level of activity of said immune cell;

wherein said CAR binds to said ligand,

wherein said ligand comprises the amino acid sequence of SEQ ID NO. 57 and optionally the amino acid sequence of SEQ ID NO. 41,

wherein said ligand is attached to the cell culture plate via passive adsorption through hydrophobic and ionic interactions.

In a still further instance, the level of activity of IFNγ secreted by the FAP CAR T-cell is determined by ELISA.

In a still further instance, the level of proliferation of the FAP CAR-T cell is determined by counting the number of viable cells by standard methods in the field.

In a further instance, the kits described herewith comprise instructions for use. The kits described herewith can also comprise appropriate buffer(s).

Methods of Screening

A further aspect provided herewith relates to the use of the method of characterizing the potency of an immune cell expressing a chimeric antigen receptor (CAR) as described herewith for screening for CARs, for instance, by carrying out the method described herewith for different CARs obtained from one donor and identify the immune cells expressing a CAR (such as CAR-T cells) which proliferate the most and optionally, if the assay is conducted on a mixture of CAR-T cells, identify the sequence of the CAR of the most abundant CAR-T cells obtained at the end of the assay.

In another aspect, the method described herewith is for screening for CAR-T cells which exhibit the highest potency.

Another aspect provided herewith relates to the use of the method of characterizing the potency of an immune cell expressing a chimeric antigen receptor (CAR) as described herewith for product characterization.

A still other aspect provided herewith relates to the use of the method of characterizing the potency of an immune cell expressing a chimeric antigen receptor (CAR) as described herewith for screening for predictive biomarkers.

A still other aspect provided herewith relates to the use of the method of characterizing the potency of an immune cell expressing a chimeric antigen receptor (CAR) as described herewith for enriching a population of immune cells into CAR-expressing immune cells.

A further aspect relates to an in vitro method of negative screening of an immune cell expressing a chimeric antigen receptor (CAR) (such as a CAR-T cell) in vitro, comprising:

(i) Providing an immune cell expressing a CAR (such as a CAR-T cell);

(ii) incubating the immune cell (such as CAR-T cell) on an antigen-specific coated support,

(iii) determining the level of activity of the stimulated immune cell (such as CAR-T cell), wherein a level of activity determined in step (iii) low or negligible is indicative of said CAR being not specific for said antigen.

Method of Enrichment

In another aspect is provided an ex vivo method of enrichment of immune cells expressing a chimeric antigen receptor (CAR), comprising:

(i) Providing a population of immune cells comprising immune cells expressing a chimeric antigen receptor targeting an antigen;

(ii) Incubating said population of cells on a ligand-coated support, wherein said support is not a cell and is not a bead and wherein said ligand comprises the antigen targeted by said CAR or a fragment of said antigen binding to said CAR;

whereby the immune cells expressing a CAR targeting said antigen, or a fragment thereof binding to said CAR, have a higher proliferation rate than immune cells that do not express said CAR, resulting in their enrichment.

In a particular aspect of the ex vivo method of enrichment described herewith, said antigen is an antigen associated with a disease state like a cancer or a viral infection.

In a still particular aspect, said antigen associated with a disease state is a tumor antigen.

If increasing the stimulation of the immune cells is needed, the incubation in step (ii) may occur in a medium containing cytokines such as IL-2, IL-7, IL-15 and/or IL-21.

The method of enrichment described herewith may be useful to provide a high proportion of immune cells expressing a particular CAR as defined herewith.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

The examples provided herewith illustrate how to carry out the potency assay described in the present application as well as some important properties of this assay, for T cells endowed with a synthetic chimeric antigen receptors (CAR) specific for CD123, CS1, CLL1, or CD22.

### Example 1. Preparation of the CAR-T Cells to be Tested

Different CAR-T cells were prepared, in view of testing their potency. In particular, CAR-T cells directed against CD123, CS1, CLL1, or CD22, called CART123, CARTCS1, CARTCLL1, and CART22, respectively, were prepared as described below. In addition to expressing one of the specified CARs, depending on the CAR-T cells, the CAR-T cells tested were genetically engineered to make them TRAC knocked-out, CD52 knocked-out, and/or B2M knocked-out and/or CS1 knocked-out, so that they could be used as effective “off-the shelf” T-cells for therapy. However, the potency assay described herewith is not dependent on these additional features of the CAR-T cells and could equally be applied to CAR-T cells which are not TRAC KO, CD52 KO, and/or B2M KO, and/or CS1 knocked-out.

Production of CART123 and CART22

Cryopreserved PBMC were thawed at 37° C., washed and re-suspended in OpTmizer medium supplemented with AB human serum (5%) for an overnight incubation at 37° C. in 5% CO₂ incubator. Then, the cells were activated with antiCD3/CD28 coated beads in OpTmizer medium supplemented with AB human serum (5%) (or 5% CTS™ Immune Cell SR) and recombinant human interleukin-2 (rhIL-2, 350 IU/ml) in a CO₂ incubator. The amplified T-cells were transduced with lentiviral particles expressing a CAR targeting CD123 or CD22 (SEQ ID NO. 13 and SEQ ID NO. 22, respectively) at MOI 5 (MOI stands for Multiplicity of Infection). 24 hours post transduction cells were cultured in OpTmizer medium supplemented either with AB human serum (5%), CTS™ Immune Cell SR (5%) rhIL-7 and rhIL-15 or with CTS™ Immune Cell SR (5%) and rhIL-2 for CART123 and CART22, respectively. 48 hours post transduction, cells were electroporated with each of the 4 mRNAs encoding both arms of TRAC_T01 TALEN (SEQ ID NO. 42 and SEQ ID NO. 43) and both arms of CD52_T01 TALEN (SEQ ID NO. 44 and SEQ ID NO. 45) using AgilePulse™ Max system. Cells were resuspended in culture medium incubated 16 to 18 h at 30° C. and expanded at 37° C. after addition of fresh culture medium and adjusting cell concentration from time to time. On the final day of culture, TCRαβ negative T cells were isolated using TCRαβ biotin and anti-biotin magnetic bead system (CliniMACS TCRα/β kit) with automated and closed magnetic support cell separation system (CliniMACS Plus Instrument and CliniMACS depletion Tubing set). After depletion, cells were resuspended in culture medium and incubated at 37° C. The next day cells were counted and centrifuged and resuspended in freezing medium (NaCl 0.45%, 20% human serum albumin solution, 22.5% dPBS and 7.5% DMSO). Cells were kept frozen until use.

Production of CARTCS1

Cryopreserved PBMC were thawed at 37° C., washed and re-suspended in Optimizer medium supplemented with AB human serum (5%) for overnight incubation at 37° C. in 5% CO₂ incubator. Cells are then activated with antiCD3/CD28 coated beads in OpTmizer medium supplemented with AB human serum (5%) and recombinant human interleukin-2 (rhIL-2, 350 IU/ml) in a CO₂ incubator (culture medium). Three days after activation the amplified T-cells were electroporated with each of the 4 mRNAs encoding both arms of TRAC_T01 TALEN (SEQ ID NO. 42 and SEQ ID NO. 43) and both arms of CS1_T01TALEN (SEQ ID NO. 48 and SEQ ID NO. 49) using AgilePulse™ Max system. Cells were incubated 16 to 18 h at 30° C. in culture medium, transferred in fresh culture medium and incubated at 37° C. 72 hours post electroporation, cells were transduced with lentiviral particles expressing a CAR targeting CS1 (SEQ ID NO. 16) at MOI 5 and cultured in OpTmizer medium supplemented with AB human serum (5%), CTS™ Immune Cell SR (5%), rhIL2 (culture medium). Cells were expanded adjusting cell concentration, from time to time. On the final day of culture TCRαβ negative cells were isolated, resuspended in culture medium for an over-night culture. The next day cells were frozen in freezing medium.

Production of CARTCLL1

Cryopreserved PBMC were thawed at 37° C., washed and re-suspended in OpTmizer medium supplemented with AB human serum (5%) for overnight incubation at 37° C. in 5% CO₂ incubator. Cells were then activated with antiCD3/CD28 coated beads in OpTmizer medium supplemented with AB human serum (5%) (or 5% CTS™ Immune Cell SR) and recombinant human interleukin-2 (rhIL-2, 350 IU/mL) in a CO₂ incubator (culture medium). Three days after activation the amplified T-cells were electroporated with each of the 4 mRNAs encoding both arms of TRAC_T01 TALEN (SEQ ID NO. 42 and SEQ ID NO. 43) and both arms of B2M_T01 TALEN (SEQ ID NO. 46 and SEQ ID NO. 47) using AgilePulse™ Max system. 1.5 hour post electroporation, cells were transduced with 3×10⁴ vg/cell of a recombinant adeno-associated virus type 6 (AAV6) vector comprising 300 bp TRAC homology arm in 5′ followed by a self-cleaving T2A peptide in frame with TRAC sequence followed by the CLL1 CAR sequence (SEQ ID NO. 19) and finally with 300 bp TRAC homology arm. Cells were expanded in the culture medium adjusting cell concentration, from time to time. On the final day of culture TCRαβ negative cells were isolated, resuspended in culture medium for an over-night culture. The next day cells were frozen in freezing medium.

TABLE 10
List of TALENs used

| Target | Target sequence | TALE-nuclease arms |
|---|---|---|
| TRAC_T01 | TTGTCCCACAGATATCC | TRAC_T01-L TALEN |
| | agaaccctgaccctg | (SEQ ID NO. 42) |
| | CCGTGTACCAGCTGAGA | TRAC_T01-R TALEN |
| | (SEQ ID NO. 50) | (SEQ ID NO. 43) |
| CD52_T01 | TTCCTCCTACTCACCAT | CD52_T01-L TALEN |
| | cagcctcctggttat | (SEQ ID NO. 44) |
| | GGTACAGGTAAGAGCAA | CD52_T01-R TALEN |
| | (SEQ ID NO. 51) | (SEQ ID NO. 45) |
| B2M_T01 | TTAGCTGTGCTCGCGCT | B2M_T01-L4 TALEN |
| | actctctctttct | (SEQ ID NO. 46) |
| | GGCCTGGAGGCTATCCA | B2M_T01-R4 TALEN |
| | (SEQ ID NO. 52) | (SEQ ID NO. 47) |
| CS1_T01 | TATATCCTTTGGCAGCT | CS1_T01-L2 TALEN |
| | cacaggtgagtcc | (SEQ ID NO. 48) |
| | GGCCGGATTCTCTTCCA | CS1_T01-R2 TALEN |
| | (SEQ ID NO. 53) | (SEQ ID NO. 49) |

### Example 2. Preparation of the Proteins to be Used for Coating

The CD123 antigen used for coating has the amino acid sequence SEQ ID NO. 25 and comprises: a leader peptide (amino acid positions 1-19), the extracellular domain of the CD123 protein (amino acid positions 20-306), a linker (amino acid positions 307-315), and the murine Fc part of IgG (amino acid positions 316-536).

The CS1 antigen used for coating has the amino acid sequence SEQ ID NO. 28 and comprises: a leader peptide (amino acid positions 1-19), the extracellular domain of the CS1 protein (amino acid positions 20-223), a linker (amino acid positions 224-232), and the murine Fc part of IgG (amino acid positions 233-453).

The CLL1 antigen used for coating has the amino acid sequence SEQ ID NO. 31 and comprises: a leader peptide (amino acid positions 1-19), the extracellular domain of the CLL1 protein (amino acid positions 20-220), a linker (amino acid positions 221-229), and the murine Fc part of IgG (amino acid positions 230-450).

The CD22 antigen used for coating has the amino acid sequence SEQ ID NO. 34 and comprises: a leader peptide (amino acid positions 1-19), the extracellular domain of the CD22 protein (amino acid positions 20-687), a linker (amino acid positions 688-696), and the murine Fc part of IgG (amino acid positions 697-916).

### Example 3. Protein Coating of a Cell Culture Dish

The protein solution for coating was prepared by diluting the protein stock solution into PBS at the required concentration (optimal coating concentration specific for each product and each application).

For CART123 for the IFNγ assay, the CD123 protein is coated at 2.75 μg/cm².

For CARTCS1, the CS1 protein is coated at 7.69 μg/cm².

For CARTCLL1, the CLL1 protein is coated at 2.75 μg/cm².

For CART22, the CD22 protein is coated at 11.00 μg/cm².

For the proliferation assays, each protein is coated at 1.10 μg/cm².

Wells of a polystyrene 24-well plate or a polystyrene 96-well plate were covered with 1 ml or 175.8 μl of the protein solution and incubated for 2 h in a humidified incubator at 37° C. and 5% CO₂. After the 2 h incubating procedure the protein solution was removed and the well washed two times with 1 ml or 175.8 μl of PBS. After the last wash, the suspension of cells to be tested is immediately pipetted onto the plates. Alternatively, the coated well plate could stay covered with PBS until the timepoint of application.

### Example 4. Determination of CAR-T Activity

Measurement of IFNγ Secretion

Wells of a 24-well plate or a 96-well plate with a polystyrene surface were coated with either CD123, CS1, CLL1, or CD22 protein as described above.

For each category of CAR-T cells tested (CART123, CARTCS1, CARTCLL1, CART22 cells), 1 to 5 (as indicated in the figure legend) million CAR-T cells/ml were pipetted in 1 ml for the 24-well plate or 175.8 μl for the 96-well plate of OpTmizer CTS onto one protein coated well. The cells were incubated on the protein coated wells for 24 h+/−1 h in a humidified incubator at 37° C. and 5% CO₂. After the 24 h incubation period, the well content was transferred into 1.5 ml microtubes, centrifuged and the supernatant transferred into a fresh 1.5 ml microtube and frozen at −80° C. After a minimum of 24 h and a maximum of three weeks the amount of IFNγ present in the supernatant was quantified using the Human IFN-gamma Quantikine ELISA Kit from R&D systems (Ref SIF50, QC281) according to provider's protocol. The measured total IFNγ was divided by the number of tested CAR-T cells to determine the maximum level of IFNγ secretion by individual cell.

Measurement of Cell Proliferation

For each category of CAR-T cells tested (CART123, CARTCS1, CARTCLL1, CART22 cells), wells of a 24-well plate were coated with their respective protein as described above in Example 3.

One million CAR-T cells were pipetted in 1 ml of OpTmizer CTS with 10% FBS onto three protein coated wells in presence of IL2 (350 IU/ml). The cells were incubated on the protein coated wells for 96 h in a humidified incubator at 37° C. and 5% CO₂. After the incubation period, the number of viable cells was quantified by Nucleocounter-200, one million cells were further transferred to an uncoated well containing fresh medium comprising IL2 (350 IU/ml). The same procedure was repeated after 48 h. The final fold expansion on each day was calculated by determining the expansion from the timepoint of seeding to the timepoint of harvest, by multiplying the number of theoretical number of cells by the fold expansion and by dividing this number by the number of initially seeded cells.

### Example 5. Specificity of the Potency Assay Described Herewith

The specificity of the activation of the CAR-T cells by their target protein was demonstrated by comparing the results obtained by incubating the different CAR-T cells tested (CART123, CARTCS1, CARTCLL1, CART22 cells) on a cell culture dish coated with (i) the target protein, (ii) no protein, or (iii) a protein comprising antigen epitopes not recognized by the CART cells («not relevant protein»).

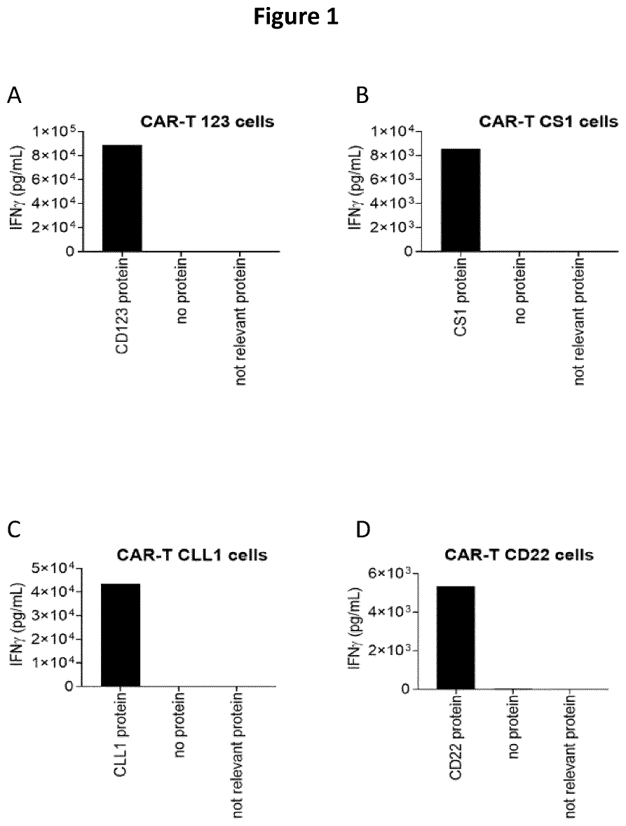

The results in FIG. 1A demonstrates that, while high IFNγ secretion was measured using the CD123 protein coated dish for CART123 cell activation, no IFNγ secretion was observed for the control conditions (not relevant protein and without stimulation).

Similar results were obtained when the assay was applied to CARTCS1, CARTCLL1, or CART22 cells with their respective target protein-coated dish (FIG. 1B-D).

These results demonstrate that the potency assay described herewith is highly specific.

### Example 6. Precision of the Potency Assay Described Herewith

Precision of the potency assay, including test for repeatability (intra assay precision of sample prepared multiple times (same day, same operator, same operating condition)) and intermediate precision (same sample tested on different days or by different operators), was evaluated.

For each of the CAR-T cells tested (CART123, CARTCS1, CARTCLL1, CART22 cells), two operators prepared 3 independent coated wells as described in Example 3 and performed IFNγ assay with 3 million CART cells.

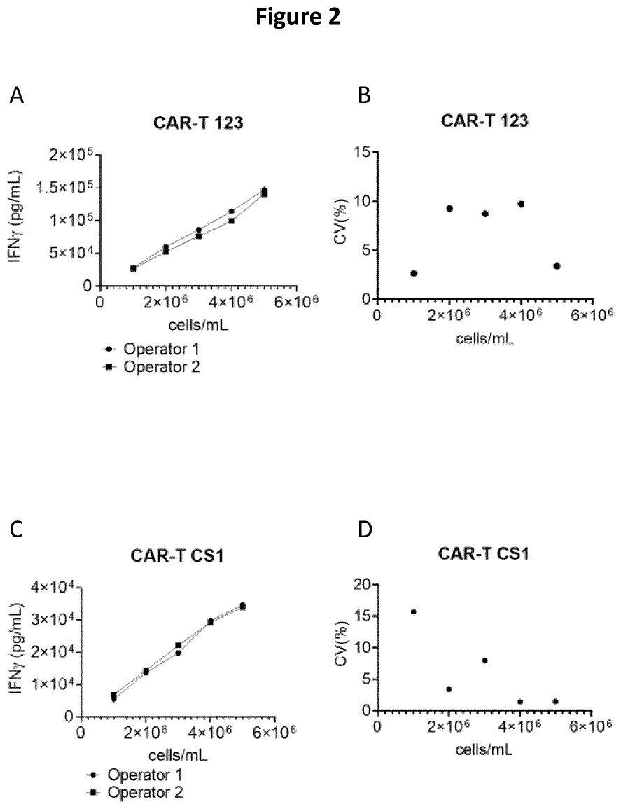

The results obtained for CART123 and CARTCS1 when the experiments were carried out by different operators are presented in FIG. 2. This shows that the test for intermediate precision between inter-Operator experiments gave a coefficient of variation of less than 17%.

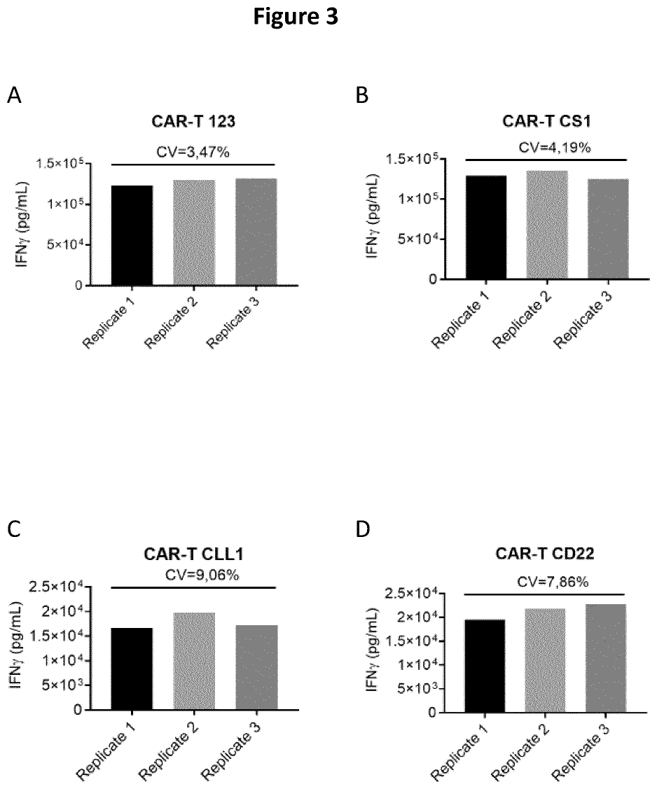

The results obtained for CART123, CARTCS1, CARTCLL1, and CART22, when an Operator repeated the experiments 3 times, are presented in FIG. 3. This shows that the test carried out between technical replicates gave a coefficient of variation of less than 10%.

Altogether, the above results demonstrate that the potency assay described herewith has a high precision for all the different CAR-T cells tested.

### Example 7. Linear Dose Response Between Activity Level and Number of CAR-T Cells in the Potency Assay Described Herewith

For each of the CAR-T cells tested (CART123, CARTCS1, CARTCLL1, CART22 cells), linearity of the potency assay was evaluated by incubating different quantities of said CAR-T cells with their target protein-coated wells for 24 hours. The level of INFγ was measured by human IFN-gamma Quantikine ELISA Kit from R&D systems according to provider's instructions.

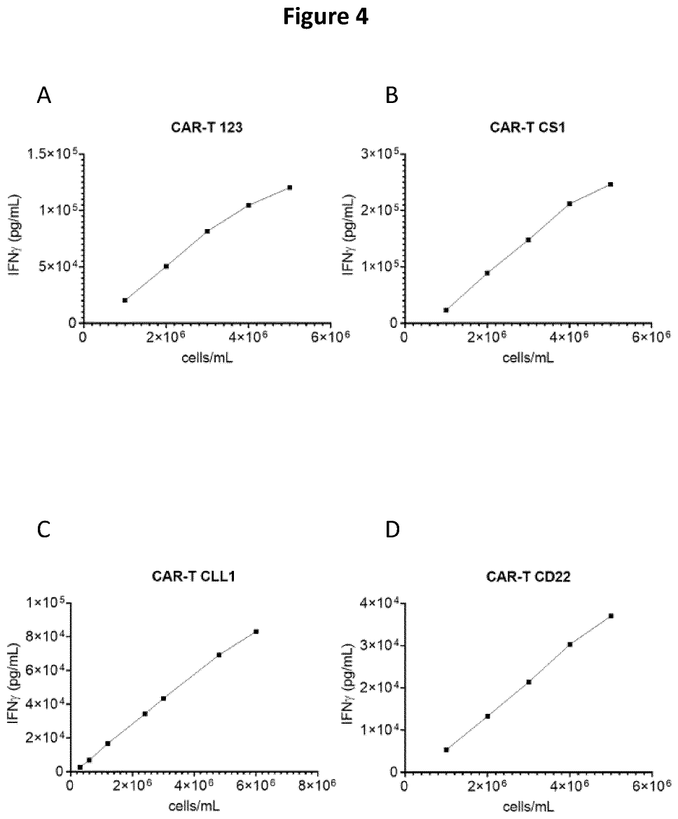

In the case of CART123, targeting CD123 protein, the results clearly demonstrate linearity between total IFNγ measurement and the number of CAR-T cells tested (FIG. 4).

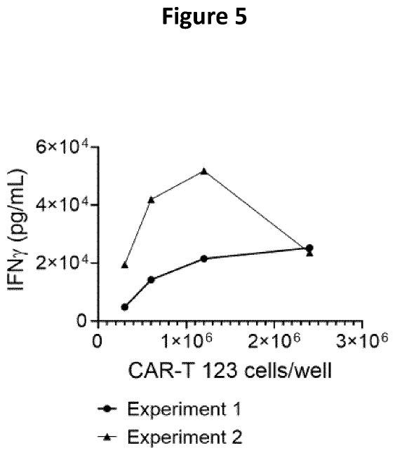

As a comparison, the linearity of the IFNγ measured in an assay using different quantities of CART123 cells incubated with 0.6 million CD123-expressing cells (target protein expressing cells instead of target protein-coated wells) was also tested. The results of this comparative assay clearly demonstrate that, when using CD123 expressing cells, it was not possible to obtain a linear dose-response (FIG. 5).

These results demonstrate the linear dose response between the activity level measured and the number of CART cells/ml of the potency assay described herewith when applied to different CART cells.

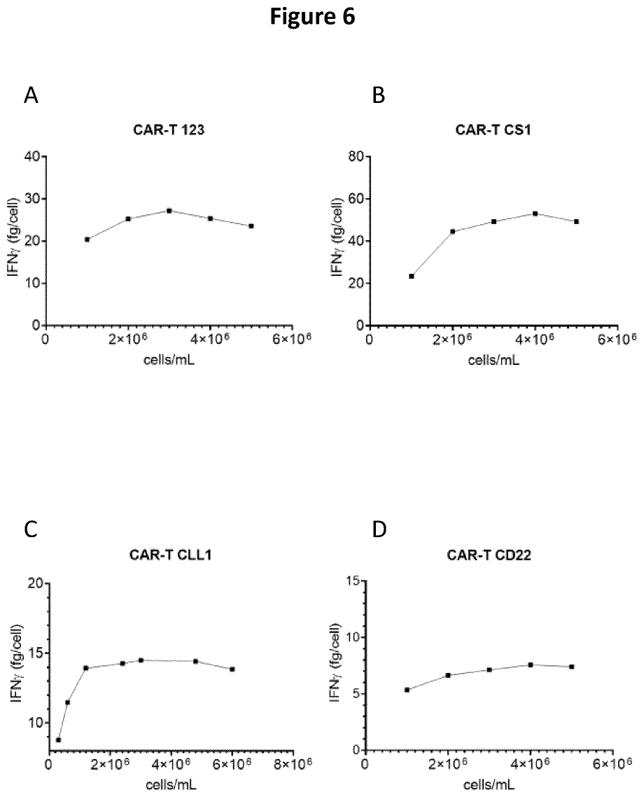

The level of secreted IFNγ per CART cell was calculated for each CART cells tested, and a maximum of IFNγ release per cell was reached, which corresponds to a plateau level in FIG. 6. The value reached at the plateau can constitute a parameter reflecting the potency of a sample. When the value is higher than a pre-determined cut-off value, it can be concluded that the sample meets the criteria to release the lot to be used for clinical application, for instance.

### Example 8. The Potency Assay Described Herewith is Useful to Compare Different Samples

To allow the comparison of activity of different batches of CAR-T cells based on the potency assay described herewith, the secreted IFNγ was measured for different CAR-T cells concentrations (1 to 5 million cells/ml) and the level of secreted IFNγ per CART cell was calculated, independently for five different batches of CART123 cells.

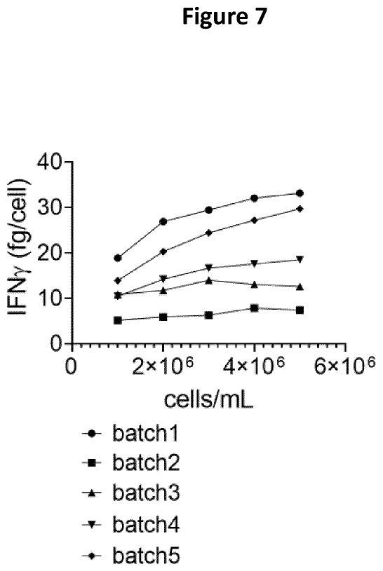

For each batch, results from one representative experiment is represented in FIG. 7.

Performing the IFNγ secretion measurement with different numbers of CART123 cells, for a given batch, showed that a maximum level of IFNγ per cell is reached, corresponding to a plateau level in FIG. 7.

Performing this test with different batches of CART123 cells showed that the different batches exhibit slight differences in the level of secreted IFNγ per CAR-T cell. This is thus possible to identify, for each tested batch, the plateau level of maximum IFNγ release per CAR-T cell, which gives a good measure for batch to batch comparison.

The determination of the maximum level of IFNg secreted per CAR-T cell, or more generally the maximum level of CAR-T activity, for each of the different samples tested with the potency assay described herewith can be used to compare those different samples and identify the one(s) exhibiting the highest level of activity.

### Example 9. Alternative Potency Assay Based on CAR-Expressing Cells Proliferation Measurement

Another possible measure for activity of the cells is the ability to proliferate after specific stimulation.

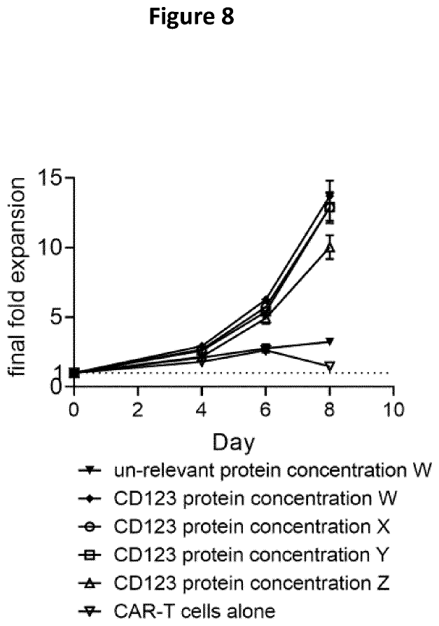

While the CAR-T cells show stagnation in proliferation in presence of IL2 and absence of any additional stimulation, they show continuous proliferation after stimulation by a CD123-protein coated surface (FIG. 8).

A range of concentrations for the coating per cm² surface was tested and showed that stimulation can be stable in a range of different protein concentrations.

1. An in vitro method of characterizing the potency of an immune cell expressing a chimeric antigen receptor (CAR), comprising:
(i) Providing an immune cell expressing a chimeric antigen receptor targeting an antigen,
(ii) Stimulating said immune cell by incubating said cell on a ligand-coated support, wherein said support is not a cell and is not a bead,
(iii) Determining the level of activity of the stimulated immune cell, wherein the ligand coated on the support in step (ii) comprises the antigen targeted by said CAR or a fragment of said antigen binding to said CAR.

2. The method of claim 1, wherein said CAR comprises an extracellular antigen-binding domain binding specifically to a target antigen associated with a disease state like a cancer or a viral infection.

3. The method of claim 2, wherein said antigen associated with a disease state is a tumor antigen.

4. The method of claim 2, wherein said antigen associated with a disease state is selected from the group consisting of CD123, CD19, CD20, CD22, CD33, 5T4, ROR1, CD38, CS1, BCMA, Flt3, CD70, EGFRvIII, WT1, HSP-70, CLL1, MUC1, ERBB2, MSLN, and FAP.

5. The method of claim 1, wherein said antigen associated with a disease state is CD123.

6. The method of claim 1, wherein said immune cell expresses two or more chimeric antigen receptors binding specifically to different target antigens associated with a cancer.

7. The method of claim 6, wherein steps (i) to (iii) are performed with one or more ligand-coated supports comprising (a) a polypeptide comprising any one of the antigens targeted by said CARs, or a fragment thereof binding to said CARs, and/or (b) two or more polypeptides comprising two or more of the antigens targeted by said CARs, or a fragment thereof binding to said CARs.

8. The method of claim 1, wherein said immune cell is selected from the group consisting of a T-cell, a NK-cell, and a macrophage.

9. The method of claim 8, wherein said immune cell is a T-cell.

10. The method of claim 1, wherein said immune cell of step (i) is an immune cell obtained from a patient or from a healthy donor, that has been engineered ex vivo to express said CAR.

11. The method of claim 1, wherein the activity which level is determined in step (iii) is cytokine secretion, degranulation, proliferation, or any combination thereof.

12. A kit for determining the potency of an immune cell expressing a chimeric antigen receptor (CAR) targeting an antigen, comprising:
(i) a ligand-coated support, wherein said support is not a cell and is not a bead,
(ii) one or more reagents for detecting the level of activity of said immune cell,
wherein said ligand comprises an antigen targeted by said CAR or a fragment of said antigen binding to said CAR.

13. The kit according to claim 12, wherein said immune cell expresses two or more CARs binding specifically to different target antigens associated with a cancer, wherein said kit comprises two or more ligand-coated supports, and wherein said ligand-coated supports independently comprise (a) a polypeptide comprising any one of the antigens targeted by said CARs, or a fragment thereof binding to said CARs, and/or (b) two or more polypeptides comprising two or more of the antigens targeted by said CARs, or a fragment thereof binding to said CARs

14. The kit according to claim 12, wherein the ligand is attached to the support by passive adsorption through hydrophobic and ionic interactions.

15. The kit according to claim 12, wherein the support is selected from the group consisting of a cell culture plate, a membrane, a matrix, a chip and a glass coverslip.

16. The kit according to claim 12 for determining the potency of an immune cell expressing a CAR targeting CD123, wherein said ligand comprises the extracellular domain of the CD123 antigen of SEQ ID NO. 24, or a fragment thereof comprising at least one epitope binding to said CAR.

17. The kit according to claim 12 for determining the potency of an immune cell expressing a CAR targeting CS1, wherein said ligand comprises the extracellular domain of the CS1 antigen of SEQ ID NO. 27, or a fragment thereof comprising at least one epitope binding to said CAR.

18. The kit according to claim 12 for determining the potency of an immune cell expressing a CAR targeting CLL1, wherein said ligand comprises the extracellular domain of the CLL1 antigen of SEQ ID NO. 30, or a fragment thereof comprising at least one epitope binding to said CAR.

19. The kit according to claim 12 for determining the potency of an immune cell expressing a CAR targeting CD22, wherein said ligand comprises the extracellular domain of the CD22 antigen of SEQ ID NO. 33, or a fragment thereof comprising at least one epitope binding to said CAR.

20. The kit according to claim 12 for determining the potency of an immune cell expressing a CAR targeting FAP, wherein said ligand comprises the extracellular domain of the FAP antigen of SEQ ID NO. 57, or a fragment thereof comprising at least one epitope binding to said CAR.
